(19) United States
(12) Patent Application Publication
(10) Pub. No.: US 2023/0357339 A1
(43) Pub. Date: Nov. 9, 2023

(54) Development of Cell-Permeable Truncated SOCS3 SH2 Domain (CP-SD) Recombinant Protein for Anti-Obesity Agent

(71) Applicant: Cellivery Therapeutics, Inc., Seoul (KR)

(72) Inventor: Daewoong Jo, Brentwood, TN (US)

(21) Appl. No.: 18/042,853

(22) PCT Filed: Sep. 3, 2021
(86) PCT No.: PCT/KR2021/011980

§ 371 Date: Feb. 24, 2023

Related U.S. Application Data
(60) Provisional application No. 63/074,703, filed on Sep. 4, 2020.

(51) Int. Cl. C07K 14/47

(52) U.S. Cl. CPC C07K 14/4703; A61K 48/00

(57) ABSTRACT

Disclosed herein is a cell-permeable truncated SOCS3 SH2 domain (CP-SD) recombinant protein. The recombinant protein inhibits the biding of SOCS3 to a leptin receptor, whereby the leptin receptor degradation induced by the biding can be suppressed. Thus, the recombinant protein can be used in a pharmaceutical composition and method for treatment and prevention of obesity, especially, leptin-resistant obesity. Moreover, the recombinant protein may be used in a pharmaceutical composition and method for treatment or prevention of obesity-related diseases including depression, intracranial hypertension, dementia, heart attack, vascular sclerosis, irregular menstruation, cancer, arthritis, asthma, fatty liver, diabetes, hyperlipidemia, high blood pressure, gallbladder disease, coronary artery disease, gout, and stroke.

### TECHNICAL FIELD

### CROSS-REFERENCE TO RELATED APPLICATIONS

This application claims the benefit of and priority to U. S. Provisional Patent Application Ser. No. 63/074,703, filled Sep. 4, 2020, the content of which is incorporated herein by reference in its entirety.

### TECHNICAL FIELD

The present invention relates to providing cell-permeable truncated SOCS3 SH2 domain (CP-SD) recombinant proteins and uses thereof for treating obesity. The truncated SH2 domain proteins from a human SOCS3 protein are cell-permeable by utilizing an advanced macromolecule transduction domain (aMTD)-based therapeutic molecule systemic delivery technology (TSDT). CP-SD recombinant proteins penetrate into cells, bind to leptin receptor and overcome leptin resistance, a key obstacle for treating obesity. The CP-SD recombinant proteins would be used as a protein-based anti-obesity agent.

### BACKGROUND ART

Leptin is an adipokine and a multi-functional cytokine which is primarily involved in regulating food intake, body weight and energy homeostasis through neuroendocrine functions. The discovery of leptin was more than two decades ago which raised great hope that an effective treatment had been found for obesity. However, leptin treatment was failed due to existence of leptin resistance in obese humans who are hyperleptinemic. Furthermore, it has been reported that exogenously administered leptin is ineffective in diet-induced obese (DIO) mice. These findings led to the notion that obesity is a condition of leptin resistance, or leptin insensitivity.

Adipocytes release leptin into the bloodstream, which binds to the leptin receptor (ObR) in the hypothalamus, across the blood-brain barrier (BBB). By binding to ObR, which is mainly expressed in the hypothalamus, leptin activates Janus-activating kinase (JAK) 2 and signal transducer and activators of transcription (STAT) 3, which subsequently stimulates the release of anorexigenic peptides such as proopiomelanocortin (POMC) and cocaine and amphetamine regulated transcript (CART) and inhibits orexigenic effects induced by neuropeptide Y (NPY) and agouti-related peptide (AgRP). Leptin signaling induces the expression of suppressor of cytokine signaling 3 (SOCS3), which is an endogenous negative feedback inhibitor. SOCS3 binds to tyrosine residues positioned at 985 on the intracellular domain of ObR via Src homology-2 (SH2) domain and suppresses leptin signaling through inhibition of JAK activity and degradation of ObR. According to the previous studies, it has been proved that increased SOCS3 expression is associated with attenuated leptin-induced activation in the arcuate nucleus (ARC) in DIO mice. In addition, recent report demonstrated that neuronal deletion of SOCS3 reduced body weight and food intake in obese mice. Evidently, these reports demonstrate a critical role of SOCS3 as a negative regulator of ObR-induced signaling in the central nervous system (CNS). The strategy to save leptin-initiated JAK/STAT signaling against SOCS3-mediated feedback regulation is for the SH2 domain of SOCS3 to competitively bind at the phosphorylated Tyr-985 of ObR against endogenous full length SOCS3, resulting in saving activated JAK/STAT signaling induced by leptin. Therefore, therapy for “blockade of excessive SOCS3-induced leptin signaling inactivation” might be useful in treating obesity.

The present disclosure describes the development of cell-permeable truncated SOCS3 SH2 domain (CP-SD) recombinant proteins through modification of the SH2 domain in human SOCS3, which are soluble and homogeneous and can overcome leptin resistance.

### REFERENCE

1. PCT/KR2016/008831

### DISCLOSURE OF INVENTION

### Technical Problem

The present inventors have made extensive efforts for structural modification of the SH2 domain of the human SOCS3 protein. The full length SH2 domain was expressed in the inclusion body, which requires a delicate refolding step in purification process for homogeneity and activity of the purified proteins. In addition, the full length SH2 domain includes a PEST motif which is not required for binding to the leptin receptor but unstablizes the SOCS3 protein. The modification of the SH2 domain based on its structural characteristics with utilizing TSDT induced development of cell-permeable truncated SOCS3 SH2 domain (CP-SD) recombinant proteins, which are homogeneous, stable and biologically active as an agent to overcome leptin resistance.

### Solution to Problem

The object of the present disclosure is to develop soluble and homogeneous recombinant proteins which can overcome leptin resistance through modifying the structure of the SH2 domain of human SOCS3.

Three systemic approaches were performed to carry out above strategy: i) the SH2 domain (G45-N185) of human SOCS3 which binds to the intracellular domain of leptin receptor, was divided based on its secondary structures to maintain its structural characteristics. The SH2 domain of human SOCS3 have residues to bind to the leptin receptor, p-Tyr binding sites including R71 and R94 which bind to the phosphorylated Y985 of the leptin receptor and receptor binding sites including G53, G54, Y127, A164, Y165, Y166, 1167 and Y168 which bind to amino acids around Y985 of the leptin receptor (FIG. 1). Three regions in the SH2 domain were selected; A β-sheet region, L69-Q96, containing R71 and R94, an α-helical region, V120-M128, containing Y127 and a β-sheet region, A164-N185, containing A164, Y165, Y166, 1167 and Y168. To increase solubility of the α-helical region, a truncate of the α-helical region was also designed, which contains H125-M128. The main cargoes from the SH2 domain consist of parts of the four regions and the regions were connected to other regions using linkers. ii) sequence-optimized advanced macromolecule transduction domain (aMTD)-enabled therapeutic molecule systemic delivery technology (TSDT) has been adopted to the cargoes, the truncates from the SH2 domain, with linkers. aMTDs which were designed based on these six critical factors (number of amino acids, bending potential, instability index, aliphatic index, hydropathy and amino acid composition), could optimize cell-permeability of cargo proteins that have therapeutic effects. Also, these aMTDs were capable of bidirectional movement across the plasma membrane as assessed by cell-to-cell protein transfer and were systemically delivered into various tissues including brain. The purified proteins with application of the two approaches showed homogeneity and binding affinity to leptin receptor. iii) solubilization domains (SDs) were additionally attached to the aMTD-fused cargoes using linkers to increase the solubility and it enables to purify the well folded cargo proteins from E. coli without a refolding step.

Through these three approaches, novel cell-permeable truncated SOCS3 SH2 domain (CP-SD) recombinant proteins were developed, which are soluble and homogeneous and overcome leptin resistance.

One aspect disclosed in the present application provides a cell-permeable truncated SOCS3 SH2 domain (CP-SD) recombinant protein:

### According to One Embodiment, the Recombinant Protein Comprises

i) a region of L69-Q96 in the SH2 domain of a human SOCS3 protein; and
ii) an advanced macromolecule transduction domain (aMTD),

wherein the aMTD has an amino acid sequence selected from the group consisting of SEQ ID Nos: 5-244.

According to one embodiment, the recombinant protein further comprises one or more region(s) selected from the group consisting of a region of V120-M128, a region of H125-M128 and a region of A164-N185 in the SH2 domain of the human SOCS3 protein.

According to one embodiment, the recombinant protein further comprises one or more solubilization domain (SD)(s).

According to one embodiment, the recombinant protein is represented by any one of the following structural formulae:

A-B,B-A,A-B-C,A-C-B,B-A-C,B-C-A,C-A-B,C-B-A and A-C-B-C

wherein A is an advanced macromolecule transduction domain (aMTD),

B is a truncated SOCS3 SH2 domain protein, and

C is a solubilization domain (SD).

According to one embodiment, the recombinant protein has an amino acid sequence selected from the group consisting of SEQ ID NOs:251-266.

According to one embodiment, the SD(s) have an amino acid sequence independently selected from the group consisting of SEQ ID NOs: 248-249.

According to one embodiment, the CP-SD recombinant protein is used for treating obesity.

Another aspect disclosed in the present application provides a polynucleotide sequence encoding the CP-SD recombinant protein.

Still another aspect disclosed in the present application provides a recombinant expression vector comprising the polynucleotide sequence.

Still another aspect disclosed in the present application provides a transformant transformed with the recombinant expression vector.

Still another aspect disclosed in the present application provides a composition comprising the CP-SD recombinant protein as an active ingredient.

Still another aspect disclosed in the present application provides a pharmaceutical composition for treating obesity, comprising the CP-SD recombinant protein as an active ingredient; and a pharmaceutically acceptable carrier.

Still another aspect disclosed in the present application provides a pharmaceutical composition for treating obesity related diseases, comprising the CP-SD recombinant protein as an active ingredient.

According to one embodiment, the obesity related diseases comprise depression, intracranial hypertension, dementia, heart attack, vascular sclerosis, irregular menstruation, cancer, arthritis, asthma, fatty liver, diabetes, hyperlipidemia, high blood pressure, gallbladder disease, coronary artery disease, gout, and stroke.

Still another aspect disclosed in the present application provides use of the CP-SD recombinant protein as a medicament for treating obesity.

Still another aspect disclosed in the present application provides use of the CP-SD recombinant protein as a medicament for treating obesity related diseases.

According to one embodiment, the obesity related diseases comprise depression, intracranial hypertension, dementia, heart attack, vascular sclerosis, irregular menstruation, cancer, arthritis, asthma, fatty liver, diabetes, hyperlipidemia, high blood pressure, gallbladder disease, coronary artery disease, gout, stroke.

Still another aspect disclosed in the present application provides a medicament comprising the CP-SD recombinant protein.

Still another aspect disclosed in the present application provides use of the CP-SD recombinant protein for the preparation of a medicament for treating obesity.

Still another aspect disclosed in the present application provides use of the CP-SD recombinant protein for the preparation of a medicament for treating obesity related diseases.

According to one embodiment, the obesity related diseases comprise depression, intracranial hypertension, dementia, heart attack, vascular sclerosis, irregular menstruation, cancer, arthritis, asthma, fatty liver, diabetes, hyperlipidemia, high blood pressure, gallbladder disease, coronary artery disease, gout, stroke.

Still another aspect disclosed in the present application provides a method of treating obesity in a subject.

The Method Comprises:

administering to the subject a therapeutically effective amount of the CP-SD recombinant protein.

Still another aspect disclosed in the present application provides a method of treating obesity related diseases in a subject.

According to one embodiment, the method comprises:

administering to the subject a therapeutically effective amount of the CP-SD recombinant protein.

According to one embodiment, the obesity related diseases comprise depression, intracranial hypertension, dementia, heart attack, vascular sclerosis, irregular menstruation, cancer, arthritis, asthma, fatty liver, diabetes, hyperlipidemia, high blood pressure, gallbladder disease, coronary artery disease, gout, stroke.

### Advantageous Effects of Invention

As disclosed in the present application, the development and establishment of CP-SD recombinant proteins, as possible therapeutics for obesity, were provided. CP-SD recombinant proteins are purified homogeneously with a simplified purification process. The proteins are cell-permeable and overcome leptin resistance, a current key obstacle for treating obesity. CP-SD recombinant proteins were also designed based on the endogenous human protein SOCS3 and it would be safety as an anti-obesity drug without side-effects.

However, the effects of the disclosures in the present application are not limited to the above-mentioned effects, and other effects not mentioned will be clearly understood by those skilled in the art from the following description.

### BRIEF DESCRIPTION OF DRAWINGS

FIG. 1a shows the mechanism of action of CP-SD.

FIG. 1b shows the structure of human SOCS3 and the residues in the SH2 domain which bind to leptin receptor.

FIG. 2a shows the structures of P2-1 and P2-1-6.

FIG. 2b shows inducible expression of aMTD524-P2-1-6 in E. coli according to example 2.

FIG. 2c shows the SDS-PAGE analysis of aMTD524-P2-1-6 purified from E. coli according to example 2.

FIG. 2d shows the size exclusion (SE)-HPLC analysis of the purified aMTD524-P2-1-6 according to example 2.

FIG. 2e shows the flow cytometry analysis of aMTD-mediated cell-permeability of FITC-labeled aMTD524-P2-1 in RAW 264.7 cells according to example 3.

FIG. 2f shows the localization of FITC-labeled aMTD524-P2-1 in RAW 264.7 cells by confocal microscopy according to example 3.

FIG. 2g shows the binding affinity of the purified aMTD524-P2-1-6 to the leptin receptor peptide with phosphorylated Y985 by isothermal titration calorimetry (ITC) according to example 4.

FIG. 2h shows overcoming leptin resistance by treatment of the purified aMTD524-P2-1-6 in leptin resistant mHypoA2/21-SOCS3 cells according to example 5.

FIG. 2i shows turbidity of purified aMTD524-P2-1-6 in storage buffer with thermal stress of 37° C. according to example 6.

FIG. 2j shows turbidity of purified aMTD524-P2-1-6 in serum free medium with thermal stress of 37° C. according to example 6.

FIG. 3a shows the structures of P8, P8-1, P9, P10, P10-1, P11, P11-1, P12, P13 and P14.

FIG. 3b shows overcoming leptin resistance by treatment of synthesized aMTD524-P8, aMTD524-P8-1, aMTD524-P9, aMTD524-P10, aMTD524-P10-1, aMTD524-P11, aMTD524-P11-1, aMTD524-P12, aMTD524-P13 and aMTD524-P14 in leptin resistant mHypoA2/21-SOCS3 cells according to example 5.

FIG. 4a shows the structure of P2-1-21.

FIG. 4b shows inducible expression of aMTD524-P2-1-21 in E. coli according to example 2.

FIG. 4c shows the SDS-PAGE analysis of aMTD524-P2-1-21 purified from E. coli according to example 2.

FIG. 4d shows the size exclusion (SE)-HPLC analysis of the purified aMTD524-P2-1-21 according to example 2.

FIG. 5a shows the structure of P2-1-23.

FIG. 5b shows inducible expression of aMTD524-P2-1-23 in E. coli according to example 2.

FIG. 5c shows the SDS-PAGE analysis of aMTD524-P2-1-23 purified from E. coli according to example 2.

FIG. 5d shows the size exclusion (SE)-HPLC analysis of the purified aMTD524-P2-1-23 according to example 2.

FIG. 6a shows the structure of P2-1-24.

FIG. 6b shows inducible expression of aMTD524-P2-1-24 in E. coli according to example 2.

FIG. 6c shows the SDS-PAGE analysis of aMTD524-P2-1-24 purified from E. coli according to example 2.

FIG. 6d shows the size exclusion (SE)-HPLC analysis of the purified aMTD524-P2-1-24 according to example 2.

FIG. 7a shows the structure of 36.

FIG. 7b shows inducible expression of aMTD524-36 in E. coli according to example 2.

FIG. 7c shows the SDS-PAGE analysis of aMTD524-36 purified from E. coli according to example 2.

FIG. 7d shows the size exclusion (SE)-HPLC analysis of the purified aMTD524-36 according to example 2.

FIG. 8a shows the structure of 37.

FIG. 8b shows inducible expression of aMTD524-37 in E. coli according to example 2.

FIG. 8c shows the SDS-PAGE analysis of aMTD524-37 purified from E. coli according to example 2.

FIG. 8d shows the size exclusion (SE)-HPLC analysis of the purified aMTD524-37 according to example 2.

FIG. 9a shows overcoming leptin resistance by treatment of the purified aMTD524-P2-1-21, aMTD524-P2-1-23, aMTD524-37 in leptin resistant mHypoA2/21-SOCS3 cells according to example 5.

FIG. 9b shows overcoming leptin resistance by treatment of the purified aMTD524-P2-1-24 in leptin resistant mHypoA2/21-SOCS3 cells according to example 5.

FIG. 9c shows overcoming leptin resistance by treatment of the purified aMTD524-36 in leptin resistant mHypoA2/21-SOCS3 cells according to example 5.

FIG. 10a shows the structure of P2-1B.

FIG. 10b shows inducible expression of aMTD524-P2-1B, aMTD343-P2-1B, aMTD385-P2-1B, aMTD485-P2-1B, aMTD830-P2-1B in E. coli according to example 2.

FIG. 10c shows the size exclusion (SE)-HPLC analysis of the purified aMTD830-P2-1B according to example 2.

FIG. 10d shows overcoming leptin resistance by treatment of the aMTD830-P2-1B in leptin resistant mHypoA2/21-SOCS3 cells according to example 5.

FIG. 10e shows turbidity of purified aMTD830-P2-1B in serum free medium with thermal stress of 37° C. according to example 6.

FIG. 11a shows the structure of P2-1E.

FIG. 11b shows inducible expression of aMTD524-P2-1E in E. coli according to example 2.

FIG. 12a shows the structure of P2-1-24B.

FIG. 12b shows inducible expression of aMTD524-P2-1-24B, aMTD343-P2-1-24B, aMTD385-P2-1-24B, aMTD485-P2-1-24B, aMTD830-P2-1-24B in E. coli according to example 2.

FIG. 12c shows the SDS-PAGE analysis of aMTD830-P2-1-24B purified from E. coli according to example 2.

FIG. 12d shows overcoming leptin resistance by treatment of the aMTD830-P2-1-24B in leptin resistant mHypoA2/21-SOCS3 cells according to example 5.

FIG. 12e shows turbidity of purified aMTD830-P2-1-24B in serum free medium with thermal stress of 37° C. according to example 6.

FIG. 13a shows the structure of P2-1-32B.

FIG. 13b shows inducible expression of aMTD524-P2-1-32B, aMTD343-P2-1-32B, aMTD385-P2-1-32B, aMTD485-P2-1-32B, aMTD830-P2-1-32B in E. coli according to example 2.

FIG. 13c shows the SDS-PAGE analysis of aMTD830-P2-1-32B purified from E. coli according to example 2.

FIG. 13d shows overcoming leptin resistance by treatment of the aMTD524-P2-1-32B, aMTD830-P2-1-32B in leptin resistant mHypoA2/21-SOCS3 cells according to example 5.

FIG. 13e shows turbidity of purified aMTD830-P2-1-32B in serum free medium with thermal stress of 37° C. according to example 6.

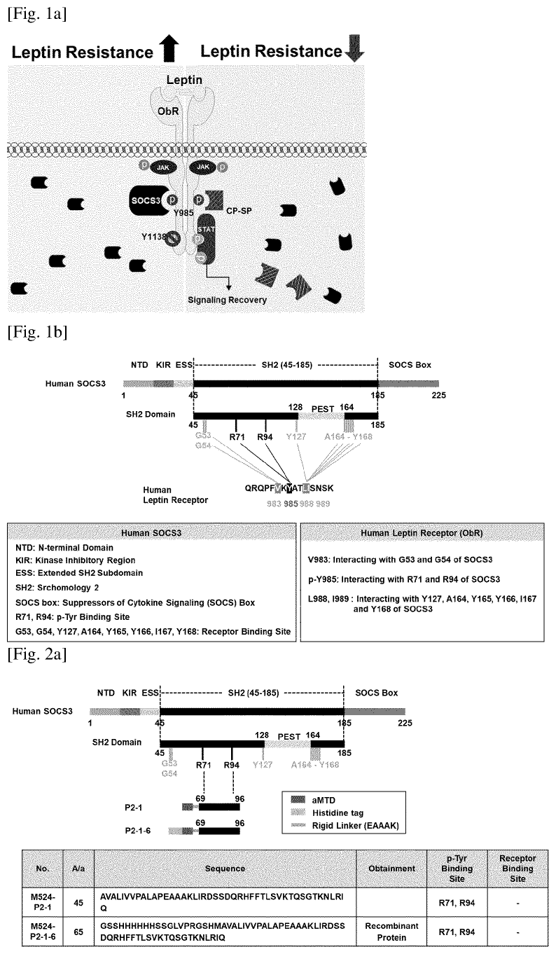

### MODE FOR THE INVENTION

Unless otherwise defined, all technical and scientific terms used herein have the same meaning as commonly understood by experts in the art to which the present invention belongs. All the publications, patents, and other documents cited in the description are incorporated by reference in their entireties.

Additionally, unless specifically stated throughout the specification, the terms “comprising”, “including”, or “containing” is intended to designate including any component (or constituent element) without particular limitations thereto, and cannot be construed as excluding the addition of a different component (or constituent element).

As used herein, the term “amino acid” is intended to encompass D-amino acids and chemically modified amino acids in a broad sense as well as naturally occurring L α-amino acids or residues thereof. For example, the amino acid mimetics and analogs fall within the scope of the amino acid. Herein, the mimetics and analogs may include functional equivalents thereof.

As used herein, the term “prevention” means all actions that are performed to suppress or delay the onset of leptin resistant obesity or obesity-related diseases by administering the cell-permeable truncated SOCS3 SH2 domain (CP-SD) recombinant protein according to the present disclosure, and the term “treatment” means all actions that are performed to alleviate or beneficially change symptoms of leptin resistant obesity or obesity-related diseases by administering the cell-permeable truncated SOCS3 SH2 domain (CP-SD) recombinant protein.

The term “administration”, as used herein, refers to the delivery of a pharmaceutical composition according to the present disclosure into a subject in any suitable manner.

As used herein, the term “subject” refers to any animal including humans, which has suffered from or is at risk for leptin resistant obesity or obesity-related diseases. Examples of the animal, which is in need of treating leptin resistant obesity- or obesity-related diseases or symptoms thereof, include cattle, horses, sheep, swine, goats, camels, antelope, dogs, and cats, but are not limited thereto.

I. CP-SD Recombinant Protein

1.SH2 Domain of SOCS3 Protein

The present disclosure provides a cell-permeable truncated SOCS3 SH2 domain (CP-SD) recombinant protein including a partial region of the SH2 domain of human SOCS3 protein. The partial region of the SH2 domain of SOCS3 protein may play an important role in binding the CP-SD recombinant protein provided according to the present disclosure to a leptin receptor.

According to one embodiment, the partial region of SH2 domain may include at least one region selected from the group consisting of L69-Q96, V120-M128, H125-M128, and A164-N185. In an exemplary embodiment, the CP-SD recombinant protein provided according to the present disclosure includes the L69-Q96 region within SH2 domain of human SOCS3 protein. In this regard, the CP-SD recombinant protein may further include at least one region selected from the group consisting of V120-M128, H125-M128, and A164-N185 within SH2 domain of human SOCS3 protein.

The L69-Q96 region within SH2 domain of SOCS3 protein has an amino acid sequence, LIRDSSDQRHFFTLSVKTQSGTKNLRIQ (SEQ ID No:1). The V120-M128 region has an amino acid sequence, VLKLVHHYM (SEQ ID No:2); the H125-M128 region has an amino acid sequence, HHYM (SEQ ID No:3); and the A164-N185 region has an amino acid sequence, AYYIYSGGEKIPLVLSRPLSSN (SEQ ID No:4). However, the SH2 domain region in the cell-permeable truncated SOCS3 SH2 domain (CP-SD) recombinant protein is not limited to the amino acid sequences of SEQ ID NOS: 1 to 4, but include any variant that exhibits an identical or similar effect to the sequences.

2.Domain that Facilitates Delivery of a Bioactive Molecule into Cells Across their Plasma Membranes

The present disclosure provides a cell-permeable truncated SOCS3 SH2 domain (CP-SD) recombinant protein comprising a domain that facilitates a bioactive molecule into cells across their plasma membranes. The domain that facilitates the delivery of a bioactive molecule into cells across their plasma membranes may be exemplified by an aMTD domain, but with no limitations thereto, and may include cationic, chimeric, hydrophobic CPP (cell penetrating peptide).

As for the bioactive molecule, their examples include proteins, peptides, nucleic acids, compounds, and so on. In the present disclosure, the aMTD domain may mean a peptide that facilitates the delivery of the above-described SH2 domain of SOCS3 protein across plasma membranes. With respect to the aMTD domain, reference may be made to Korean Patent Number 10-1971021, the content of which is incorporated herein by reference in its entirety.

In one embodiment, the aMTD domain may include the amino acid sequence selected from the group SEQ ID NOS: 5 to 244. In an exemplary embodiment, the aMTD domain may include the amino acid sequence selected from SEQ ID NOS: 85, 95, 118, 126, and 197.

TABLE 1
aMTD Domain

| SEQ | |
|---|---|
| ID | |
| No. | Sequence |
| 5 | Ala Ala Ala Leu Ala Pro Val Val Leu Ala Leu Pro |
| 6 | Ala Ala Ala Val Pro Leu Leu Ala Val Val Val Pro |
| 7 | Ala Ala Leu Leu Val Pro Ala Ala Val Leu Ala Pro |
| 8 | Ala Leu Ala Leu Leu Pro Val Ala Ala Leu Ala Pro |
| 9 | Ala Ala Ala Leu Leu Pro Val Ala Leu Val Ala Pro |
| 10 | Val Val Ala Leu Ala Pro Ala Leu Ala Ala Leu Pro |
| 11 | Leu Leu Ala Ala Val Pro Ala Val Leu Leu Ala Pro |
| 12 | Ala Ala Ala Leu Val Pro Val Val Ala Leu Leu Pro |
| 13 | Ala Val Ala Leu Leu Pro Ala Leu Leu Ala Val Pro |
| 14 | Ala Val Val Leu Val Pro Val Leu Ala Ala Ala Pro |
| 15 | Val Val Leu Val Leu Pro Ala Ala Ala Ala Val Pro |
| 16 | Ile Ala Leu Ala Ala Pro Ala Leu Ile Val Ala Pro |
| 17 | Ile Val Ala Val Ala Pro Ala Leu Val Ala Leu Pro |
| 18 | Val Ala Ala Leu Pro Val Val Ala Val Val Ala Pro |
| 19 | Leu Leu Ala Ala Pro Leu Val Val Ala Ala Val Pro |
| 20 | Ala Leu Ala Val Pro Val Ala Leu Leu Val Ala Pro |
| 21 | Val Ala Ala Leu Pro Val Leu Leu Ala Ala Leu Pro |
| 22 | Val Ala Leu Leu Ala Pro Val Ala Leu Ala Val Pro |
| 23 | Ala Ala Leu Leu Val Pro Ala Leu Val Ala Val Pro |
| 24 | Ala Ile Val Ala Leu Pro Val Ala Val Leu Ala Pro |
| 25 | Ile Ala Ile Val Ala Pro Val Val Ala Leu Ala Pro |
| 26 | Ala Ala Leu Leu Pro Ala Leu Ala Ala Leu Leu Pro |
| 27 | Ala Val Val Leu Ala Pro Val Ala Ala Val Leu Pro |
| 28 | Leu Ala Val Ala Ala Pro Leu Ala Leu Ala Leu Pro |
| 29 | Ala Ala Val Ala Ala Pro Leu Leu Leu Ala Leu Pro |
| 30 | Leu Leu Val Leu Pro Ala Ala Ala Leu Ala Ala Pro |
| 31 | Leu Val Ala Leu Ala Pro Val Ala Ala Val Leu Pro |
| 32 | Leu Ala Leu Ala Pro Ala Ala Leu Ala Leu Leu Pro |
| 33 | Ala Leu Ile Ala Ala Pro Ile Leu Ala Leu Ala Pro |
| 34 | Ala Val Val Ala Ala Pro Leu Val Leu Ala Leu Pro |
| 35 | Leu Leu Ala Leu Ala Pro Ala Ala Leu Leu Ala Pro |
| 36 | Ala Ile Val Ala Leu Pro Ala Leu Ala Leu Ala Pro |
| 37 | Ala Ala Ile Ile Val Pro Ala Ala Leu Leu Ala Pro |
| 38 | Ile Ala Val Ala Leu Pro Ala Leu Ile Ala Ala Pro |
| 39 | Ala Val Ile Val Leu Pro Ala Leu Ala Val Ala Pro |
| 40 | Ala Val Leu Ala Val Pro Ala Val Leu Val Ala Pro |
| 41 | Val Leu Ala Ile Val Pro Ala Val Ala Leu Ala Pro |
| 42 | Leu Leu Ala Val Val Pro Ala Val Ala Leu Ala Pro |
| 43 | Ala Val Ile Ala Leu Pro Ala Leu Ile Ala Ala Pro |
| 44 | Ala Val Val Ala Leu Pro Ala Ala Leu Ile Val Pro |
| 45 | Leu Ala Leu Val Leu Pro Ala Ala Leu Ala Ala Pro |
| 46 | Leu Ala Ala Val Leu Pro Ala Leu Leu Ala Ala Pro |
| 47 | Ala Leu Ala Val Pro Val Ala Leu Ala Ile Val Pro |
| 48 | Ala Leu Ile Ala Pro Val Val Ala Leu Val Ala Pro |
| 49 | Leu Leu Ala Ala Pro Val Val Ile Ala Leu Ala Pro |
| 50 | Leu Ala Ala Ile Val Pro Ala Ile Ile Ala Val Pro |
| 51 | Ala Ala Leu Val Leu Pro Leu Ile Ile Ala Ala Pro |
| 52 | Leu Ala Leu Ala Val Pro Ala Leu Ala Ala Leu Pro |
| 53 | Leu Ile Ala Ala Leu Pro Ala Val Ala Ala Leu Pro |
| 54 | Ala Leu Ala Leu Val Pro Ala Ile Ala Ala Leu Pro |
| 55 | Ala Ala Ile Leu Ala Pro Ile Val Ala Leu Ala Pro |
| 56 | Ala Leu Leu Ile Ala Pro Ala Ala Val Ile Ala Pro |
| 57 | Ala Ile Leu Ala Val Pro Ile Ala Val Val Ala Pro |
| 58 | Ile Leu Ala Ala Val Pro Ile Ala Leu Ala Ala Pro |
| 59 | Val Ala Ala Leu Leu Pro Ala Ala Ala Val Leu Pro |
| 60 | Ala Ala Ala Val Val Pro Val Leu Leu Val Ala Pro |
| 61 | Ala Ala Leu Leu Val Pro Ala Leu Val Ala Ala Pro |
| 62 | Ala Ala Val Leu Leu Pro Val Ala Leu Ala Ala Pro |
| 63 | Ala Ala Ala Leu Ala Pro Val Leu Ala Leu Val Pro |
| 64 | Leu Val Leu Val Pro Leu Leu Ala Ala Ala Ala Pro |
| 65 | Ala Leu Ile Ala Val Pro Ala Ile Ile Val Ala Pro |
| 66 | Ala Leu Ala Val Ile Pro Ala Ala Ala Ile Leu Pro |
| 67 | Leu Ala Ala Ala Pro Val Val Ile Val Ile Ala Pro |
| 68 | Val Leu Ala Ile Ala Pro Leu Leu Ala Ala Val Pro |
| 69 | Ala Leu Ile Val Leu Pro Ala Ala Val Ala Val Pro |
| 70 | Val Leu Ala Val Ala Pro Ala Leu Ile Val Ala Pro |
| 71 | Ala Ala Leu Leu Ala Pro Ala Leu Ile Val Ala Pro |
| 72 | Ala Leu Ile Ala Pro Ala Val Ala Leu Ile Val Pro |
| 73 | Ala Ile Val Leu Leu Pro Ala Ala Val Val Ala Pro |
| 74 | Val Ile Ala Ala Pro Val Leu Ala Val Leu Ala Pro |
| 75 | Leu Ala Leu Ala Pro Ala Leu Ala Leu Leu Ala Pro |
| 76 | Ala Ile Ile Leu Ala Pro Ile Ala Ala Ile Ala Pro |
| 77 | Ile Ala Leu Ala Ala Pro Ile Leu Leu Ala Ala Pro |
| 78 | Ile Val Ala Val Ala Leu Pro Ala Leu Ala Val Pro |
| 79 | Val Val Ala Ile Val Leu Pro Ala Leu Ala Ala Pro |
| 80 | Ile Val Ala Val Ala Leu Pro Val Ala Leu Ala Pro |
| 81 | Ile Val Ala Val Ala Leu Pro Ala Ala Leu Val Pro |
| 82 | Ile Val Ala Val Ala Leu Pro Ala Val Ala Leu Pro |
| 83 | Ile Val Ala Val Ala Leu Pro Ala Val Leu Ala Pro |
| 84 | Val Ile Val Ala Leu Ala Pro Ala Val Leu Ala Pro |
| 85 | Ile Val Ala Val Ala Leu Pro Ala Leu Val Ala Pro |
| 86 | Ala Leu Leu Ile Val Ala Pro Val Ala Val Ala Pro |
| 87 | Ala Val Val Ile Val Ala Pro Ala Val Ile Ala Pro |
| 88 | Ala Val Leu Ala Val Ala Pro Ala Leu Ile Val Pro |
| 89 | Leu Val Ala Ala Val Ala Pro Ala Leu Ile Val Pro |
| 90 | Ala Val Ile Val Val Ala Pro Ala Leu Leu Ala Pro |
| 91 | Val Val Ala Ile Val Leu Pro Ala Val Ala Ala Pro |
| 92 | Ala Ala Ala Leu Val Ile Pro Ala Ile Leu Ala Pro |
| 93 | Val Ile Val Ala Leu Ala Pro Ala Leu Leu Ala Pro |
| 94 | Val Ile Val Ala Ile Ala Pro Ala Leu Leu Ala Pro |
| 95 | Ile Val Ala Ile Ala Val Pro Ala Leu Val Ala Pro |
| 96 | Ala Ala Leu Ala Val Ile Pro Ala Ala Ile Leu Pro |
| 97 | Ala Leu Ala Ala Val Ile Pro Ala Ala Ile Leu Pro |
| 98 | Ala Ala Ala Leu Val Ile Pro Ala Ala Ile Leu Pro |
| 99 | Leu Ala Ala Ala Val Ile Pro Ala Ala Ile Leu Pro |
| 100 | Leu Ala Ala Ala Val Ile Pro Val Ala Ile Leu Pro |
| 101 | Ala Ala Ile Leu Ala Ala Pro Leu Ile Ala Val Pro |
| 102 | Val Val Ala Ile Leu Ala Pro Leu Leu Ala Ala Pro |
| 103 | Ala Val Val Val Ala Ala Pro Val Leu Ala Leu Pro |
| 104 | Ala Val Val Ala Ile Ala Pro Val Leu Ala Leu Pro |
| 105 | Ala Leu Ala Ala Leu Val Pro Ala Val Leu Val Pro |
| 106 | Ala Leu Ala Ala Leu Val Pro Val Ala Leu Val Pro |
| 107 | Leu Ala Ala Ala Leu Val Pro Val Ala Leu Val Pro |
| 108 | Ala Leu Ala Ala Leu Val Pro Ala Leu Val Val Pro |
| 109 | Ile Ala Ala Val Ile Val Pro Ala Val Ala Leu Pro |
| 110 | Ile Ala Ala Val Leu Val Pro Ala Val Ala Leu Pro |
| 111 | Ala Val Ala Ile Leu Val Pro Leu Leu Ala Ala Pro |
| 112 | Ala Val Val Ile Leu Val Pro Leu Ala Ala Ala Pro |
| 113 | Ile Ala Ala Val Ile Val Pro Val Ala Ala Leu Pro |
| 114 | Ala Ile Ala Ile Ala Ile Val Pro Val Ala Leu Pro |
| 115 | Ile Leu Ala Val Ala Ala Ile Pro Val Ala Val Pro |
| 116 | Ile Leu Ala Ala Ala Ile Ile Pro Ala Ala Leu Pro |
| 117 | Leu Ala Val Val Leu Ala Ala Pro Ala Ile Val Pro |
| 118 | Ala Ile Leu Ala Ala Ile Val Pro Leu Ala Val Pro |
| 119 | Val Ile Val Ala Leu Ala Val Pro Ala Leu Ala Pro |
| 120 | Ala Ile Val Ala Leu Ala Val Pro Val Leu Ala Pro |
| 121 | Ala Ala Ile Ile Ile Val Leu Pro Ala Ala Leu Pro |
| 122 | Leu Ile Val Ala Leu Ala Val Pro Ala Leu Ala Pro |
| 123 | Ala Ile Ile Ile Val Ile Ala Pro Ala Ala Ala Pro |
| 124 | Leu Ala Ala Leu Ile Val Val Pro Ala Val Ala Pro |
| 125 | Ala Leu Leu Val Ile Ala Val Pro Ala Val Ala Pro |
| 126 | Ala Val Ala Leu Ile Val Val Pro Ala Leu Ala Pro |
| 127 | Ala Leu Ala Ile Val Val Ala Pro Val Ala Val Pro |
| 128 | Leu Leu Ala Leu Ile Ile Ala Pro Ala Ala Ala Pro |
| 129 | Ala Leu Ala Leu Ile Ile Val Pro Ala Val Ala Pro |
| 130 | Leu Leu Ala Ala Leu Ile Ala Pro Ala Ala Leu Pro |
| 131 | Ile Val Ala Leu Ile Val Ala Pro Ala Ala Val Pro |
| 132 | Val Val Leu Val Leu Ala Ala Pro Ala Ala Val Pro |
| 133 | Ala Ala Val Ala Ile Val Leu Pro Ala Val Val Pro |
| 134 | Ala Leu Ile Ala Ala Ile Val Pro Ala Leu Val Pro |
| 135 | Ala Leu Ala Val Ile Val Val Pro Ala Leu Ala Pro |
| 136 | Val Ala Ile Ala Leu Ile Val Pro Ala Leu Ala Pro |
| 137 | Val Ala Ile Val Leu Val Ala Pro Ala Val Ala Pro |
| 138 | Val Ala Val Ala Leu Ile Val Pro Ala Leu Ala Pro |
| 139 | Ala Val Ile Leu Ala Leu Ala Pro Ile Val Ala Pro |
| 140 | Ala Leu Ile Val Ala Ile Ala Pro Ala Leu Val Pro |
| 141 | Ala Ala Ile Leu Ile Ala Val Pro Ile Ala Ala Pro |
| 142 | Val Ile Val Ala Leu Ala Ala Pro Val Leu Ala Pro |
| 143 | Val Leu Val Ala Leu Ala Ala Pro Val Ile Ala Pro |
| 144 | Val Ala Leu Ile Ala Val Ala Pro Ala Val Val Pro |
| 145 | Val Ile Ala Ala Val Leu Ala Pro Val Ala Val Pro |
| 146 | Ala Leu Ile Val Leu Ala Ala Pro Val Ala Val Pro |
| 147 | Val Ala Ala Ala Ile Ala Leu Pro Ala Ile Val Pro |
| 148 | Ile Leu Ala Ala Ala Ala Ala Pro Leu Ile Val Pro |
| 149 | Leu Ala Leu Val Leu Ala Ala Pro Ala Ile Val Pro |
| 150 | Ala Leu Ala Val Val Ala Leu Pro Ala Ile Val Pro |
| 151 | Ala Ala Ile Leu Ala Pro Ile Val Ala Ala Leu Pro |
| 152 | Ile Leu Ile Ala Ile Ala Ile Pro Ala Ala Ala Pro |
| 153 | Leu Ala Ile Val Leu Ala Ala Pro Val Ala Val Pro |
| 154 | Ala Ala Ile Ala Ile Ile Ala Pro Ala Ile Val Pro |
| 155 | Leu Ala Val Ala Ile Val Ala Pro Ala Leu Val Pro |
| 156 | Leu Ala Ile Val Leu Ala Ala Pro Ala Val Leu Pro |
| 157 | Ala Ala Ile Val Leu Ala Leu Pro Ala Val Leu Pro |
| 158 | Ala Leu Leu Val Ala Val Leu Pro Ala Ala Leu Pro |
| 159 | Ala Ala Leu Val Ala Val Leu Pro Val Ala Leu Pro |
| 160 | Ala Ile Leu Ala Val Ala Leu Pro Leu Leu Ala Pro |
| 161 | Ile Val Ala Val Ala Leu Val Pro Ala Leu Ala Pro |
| 162 | Ile Val Ala Val Ala Leu Leu Pro Ala Leu Ala Pro |
| 163 | Ile Val Ala Val Ala Leu Leu Pro Ala Val Ala Pro |
| 164 | Ile Val Ala Leu Ala Val Leu Pro Ala Val Ala Pro |
| 165 | Val Ala Val Leu Ala Val Leu Pro Ala Leu Ala Pro |
| 166 | Ile Ala Val Leu Ala Val Ala Pro Ala Val Leu Pro |
| 167 | Leu Ala Val Ala Ile Ile Ala Pro Ala Val Ala Pro |
| 168 | Val Ala Leu Ala Ile Ala Leu Pro Ala Val Leu Pro |
| 169 | Ala Ile Ala Ile Ala Leu Val Pro Val Ala Leu Pro |
| 170 | Ala Ala Val Val Ile Val Ala Pro Val Ala Leu Pro |
| 171 | Val Ala Ile Ile Val Val Ala Pro Ala Leu Ala Pro |
| 172 | Val Ala Leu Leu Ala Ile Ala Pro Ala Leu Ala Pro |
| 173 | Val Ala Val Leu Ile Ala Val Pro Ala Leu Ala Pro |
| 174 | Ala Val Ala Leu Ala Val Leu Pro Ala Val Val Pro |
| 175 | Ala Val Ala Leu Ala Val Val Pro Ala Val Leu Pro |
| 176 | Ile Val Val Ile Ala Val Ala Pro Ala Val Ala Pro |
| 177 | Ile Val Val Ala Ala Val Val Pro Ala Leu Ala Pro |
| 178 | Ile Val Ala Leu Val Pro Ala Val Ala Ile Ala Pro |
| 179 | Val Ala Ala Leu Pro Ala Val Ala Leu Val Val Pro |
| 180 | Leu Val Ala Ile Ala Pro Leu Ala Val Leu Ala Pro |
| 181 | Ala Val Ala Leu Val Pro Val Ile Val Ala Ala Pro |
| 182 | Ala Ile Ala Val Ala Ile Ala Pro Val Ala Leu Pro |
| 183 | Ala Ile Ala Leu Ala Val Pro Val Leu Ala Leu Pro |
| 184 | Leu Val Leu Ile Ala Ala Ala Pro Ile Ala Leu Pro |
| 185 | Leu Val Ala Leu Ala Val Pro Ala Ala Val Leu Pro |
| 186 | Ala Val Ala Leu Ala Val Pro Ala Leu Val Leu Pro |
| 187 | Leu Val Val Leu Ala Ala Ala Pro Leu Ala Val Pro |
| 188 | Leu Ile Val Leu Ala Ala Pro Ala Leu Ala Ala Pro |
| 189 | Val Ile Val Leu Ala Ala Pro Ala Leu Ala Ala Pro |
| 190 | Ala Val Val Leu Ala Val Pro Ala Leu Ala Val Pro |
| 191 | Leu Ile Ile Val Ala Ala Ala Pro Ala Val Ala Pro |
| 192 | Ile Val Ala Val Ile Val Ala Pro Ala Val Ala Pro |
| 193 | Leu Val Ala Leu Ala Ala Pro Ile Ile Ala Val Pro |
| 194 | Ile Ala Ala Val Leu Ala Ala Pro Ala Leu Val Pro |
| 195 | Ile Ala Leu Leu Ala Ala Pro Ile Ile Ala Val Pro |
| 196 | Ala Ala Leu Ala Leu Val Ala Pro Val Ile Val Pro |
| 197 | Ile Ala Leu Val Ala Ala Pro Val Ala Leu Val Pro |
| 198 | Ile Ile Val Ala Val Ala Pro Ala Ala Ile Val Pro |
| 199 | Ala Val Ala Ala Ile Val Pro Val Ile Val Ala Pro |
| 200 | Ala Val Leu Val Leu Val Ala Pro Ala Ala Ala Pro |
| 201 | Val Val Ala Leu Leu Ala Pro Leu Ile Ala Ala Pro |
| 202 | Ala Ala Val Val Ile Ala Pro Leu Leu Ala Val Pro |
| 203 | Ile Ala Val Ala Val Ala Ala Pro Leu Leu Val Pro |
| 204 | Leu Val Ala Ile Val Val Leu Pro Ala Val Ala Pro |
| 205 | Ala Val Ala Ile Val Val Leu Pro Ala Val Ala Pro |
| 206 | Ala Val Ile Leu Leu Ala Pro Leu Ile Ala Ala Pro |
| 207 | Leu Val Ile Ala Leu Ala Ala Pro Val Ala Leu Pro |
| 208 | Val Leu Ala Val Val Leu Pro Ala Val Ala Leu Pro |
| 209 | Val Leu Ala Val Ala Ala Pro Ala Val Leu Leu Pro |
| 210 | Ala Ala Val Val Leu Leu Pro Ile Ile Ala Ala Pro |
| 211 | Ala Leu Leu Val Ile Ala Pro Ala Ile Ala Val Pro |
| 212 | Ala Val Leu Val Ile Ala Val Pro Ala Ile Ala Pro |
| 213 | Ala Leu Leu Val Val Ile Ala Pro Leu Ala Ala Pro |
| 214 | Val Leu Val Ala Ala Ile Leu Pro Ala Ala Ile Pro |
| 215 | Val Leu Val Ala Ala Val Leu Pro Ile Ala Ala Pro |
| 216 | Val Leu Ala Ala Ala Val Leu Pro Leu Val Val Pro |
| 217 | Ala Ile Ala Ile Val Val Pro Ala Val Ala Val Pro |
| 218 | Val Ala Ile Ile Ala Val Pro Ala Val Val Ala Pro |
| 219 | Ile Val Ala Leu Val Ala Pro Ala Ala Val Val Pro |
| 220 | Ala Ala Ile Val Leu Leu Pro Ala Val Val Val Pro |
| 221 | Ala Ala Leu Ile Val Val Pro Ala Val Ala Val Pro |
| 222 | Ala Ile Ala Leu Val Val Pro Ala Val Ala Val Pro |
| 223 | Leu Ala Ile Val Pro Ala Ala Ile Ala Ala Leu Pro |
| 224 | Leu Val Ala Ile Ala Pro Ala Val Ala Val Leu Pro |
| 225 | Val Leu Ala Val Ala Pro Ala Val Ala Val Leu Pro |
| 226 | Ile Leu Ala Val Val Ala Ile Pro Ala Ala Ala Pro |
| 227 | Ile Leu Val Ala Ala Ala Pro Ile Ala Ala Leu Pro |
| 228 | Ile Leu Ala Val Ala Ala Ile Pro Ala Ala Leu Pro |
| 229 | Val Ile Ala Ile Pro Ala Ile Leu Ala Ala Ala Pro |
| 230 | Ala Ile Ile Ile Val Val Pro Ala Ile Ala Ala Pro |
| 231 | Ala Ile Leu Ile Val Val Ala Pro Ile Ala Ala Pro |
| 232 | Ala Val Ile Val Pro Val Ala Ile Ile Ala Ala Pro |
| 233 | Ala Val Val Ile Ala Leu Pro Ala Val Val Ala Pro |
| 234 | Ala Leu Val Ala Val Ile Ala Pro Val Val Ala Pro |
| 235 | Ala Leu Val Ala Val Leu Pro Ala Val Ala Val Pro |
| 236 | Ala Leu Val Ala Pro Leu Leu Ala Val Ala Val Pro |
| 237 | Ala Val Leu Ala Val Val Ala Pro Val Val Ala Pro |
| 238 | Ala Val Ile Ala Val Ala Pro Leu Val Val Ala Pro |
| 239 | Ala Val Ile Ala Leu Ala Pro Val Val Val Ala Pro |
| 240 | Val Ala Ile Ala Leu Ala Pro Val Val Val Ala Pro |
| 241 | Val Ala Leu Ala Leu Ala Pro Val Val Val Ala Pro |
| 242 | Val Ala Ala Leu Leu Pro Ala Val Val Val Ala Pro |
| 243 | Val Ala Leu Ala Leu Pro Ala Val Val Val Ala Pro |
| 244 | Val Ala Leu Leu Ala Pro Ala Val Val Val Ala Pro |

3.Linker

The CP-SD recombinant protein provided according to the present disclosure may further comprise a linker in addition to the partial region of SH2 domain and the aMTD domain. In one embodiment, the CP-SD recombinant protein may include a linker within the partial region of SH2 domain, within the aMTD, between the partial region of SH2 domain and the aMTD domain, or between the partial region of SH2 domain and a solubilization domain. Below, a description will be given of the solubilization domain.

The linker is intended to encompass a rigid linker or a flexible linker. In one embodiment, the linker may include an amino acid sequence composed of 2 to 8, 3 to 7, 4 to 6, or 5 amino acid residues. In an exemplary embodiment, the linker may include an amino acid sequence composed of 5 amino acid residues, playing a role in linking the partial region of SH2 domain and the aMTD domain. In a more exemplary embodiment, the linker may be composed of the amino acid sequence of EAAAK (SEQ ID NO: 245), GGGGS (SEQ ID NO: 246), or GSGS (SEQ ID NO: 247). In this regard, the linker may include 2 or more, 3 or more, 4 or mor, 5 or more, 6 or more, 7 or more, 8 or more, 9 or more, or 10 or more repeats of the amino acid sequence of SEQ ID NO: 245, 246, or 247 according to an embodiment of the present disclosure. However, no limitations are imparted to the linker. So long as it is known to be suitable for binding a peptide in the art, any linker may be used in the present disclosure.

4.Solubilization domain

The CP-SD recombinant protein provided according to the present disclosure may further comprise at least one solubilization domain in addition to a partial region of SH2 domain and an aMTD domain. In one embodiment, the CP-SD recombinant protein may comprise a partial region of SH2 domain, an aMTD domain, and a solubilization domain. In another embodiment, the CP-SD recombinant protein may comprise a partial region of SH2 domain, an aMTD domain, a linker, and a solubilization domain.

Given a solubilization domain, the CP-SD recombinant protein of the present disclosure enjoys the advantage of improving in in-vivo solubility. In one embodiment, the solubilization domain includes a peptide that acts to increase solubility of a bioactive molecule. In an exemplary embodiment, the solubilization domain may include an amino acid sequence selected from the group consisting of MAEQSDKD VKYYTLEEIQKHKDSKSTWLILHHKVYDLTKFLEEHPGGEEVLGEQAGGDAT ENFEDVGHSTDARELSKTYIIGELHPDDRSKIAKPSETL (SEQ ID No: 248) and GSLQDSEVNQEAKPEVKPEVKPETHINLKVSDGSSEIFFKIKKTTPLRRLMEAF AKRQGKEMDSLTFLYDGIEIQADQTPEDLDMEDNDIIEAHREQIGG (SEQ ID No: 249). However, the solubilization domain is not limited thereto and may be any domain that is known to increase solubility of a bioactive molecule.

5.Histidine Tag

The CP-SD recombinant protein provided according to the present disclosure may comprise a histidine tag. In one embodiment, the histidine tag may be fused to one end of the CP-SD recombinant protein. In this context, the CP-SD recombinant protein may comprise a partial region of SH2 domain and an aMTD domain according to an embodiment. In another embodiment, the CP-SD recombinant protein may comprise a partial region of SH2 domain, an aMTD domain, and a linker. In another embodiment, the CP-SD recombinant protein may comprise a partial region of SH2 domain, an aMTD domain, a linker, and a solubilization domain.

According to an embodiment, the histidine tag may include the amino acid sequence of GSSHHHHHHSSGLVPRGSHM (SEQ ID No:250).

6.Ligand

The CP-SD recombinant protein provided according to the present disclosure may further comprise a ligand binding selectively to a receptor on specific cells, tissues, or organs so that the recombinant protein can be properly delivered into the cells, tissues, or organs. The CP-SD recombinant protein comprising the ligand can be more effectively delivered to a target site through the ligand.

### II. Specific Example of CP-SD Recombinant Protein

The CP-SD recombinant protein provided according to the present disclosure can be represented by a structural formula selected from among A-B, B-A, A-B-C, A-C-B, B-A-C, B-C-A, C-A-B, C-B-A, and A-C-B-C. In the structural formulas, A is an aMTD domain, B is a portion of SH2 domain in SOCS3, and C is a solubilization domain. In the recombinant protein, one domain may be linked to an adjacent domain via a linker, but not necessarily.

In an exemplary embodiment, the CP-SD recombinant protein provided according to the present disclosure may include one of the following amino acid sequences. However, the amino acid sequence of the CP-SD recombinant protein is not limited thereto, but may be any sequence that is possible from the combinations described in section I. CP-SD recombinant protein.

TABLE 2
Specific example of CP-SD recombinant protein

| | | SEQ |
|---|---|---|
| | | ID |
| No. | Sequence | No. |
| aMTD524-P2- | AVALIVVPALAPEAAAKLIRDSSDQ | 251 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD343-P2- | IVAVALPALVAPEAAAKLIRDSSDQ | 252 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD385-P2- | IVAIAVPALVAPEAAAKLIRDSSDQ | 253 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD485-P2- | AILAAIVPLAVPEAAAKLIRDSSDQ | 254 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD830-P2- | IALVAAPVALVPEAAAKLIRDSSDQ | 255 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD524-P2- | AVALIVVPALAPEAAAKLIRDSSDQ | 256 |
| 1E | RHFFTLSVKTQSGTKNLRIQEAAAK | |
| | EAAAKEAAAKGSLQDSEVNQEAKPE | |
| | VKPEVKPETHINLKVSDGSSEIFFK | |
| | IKKTTPLRRLMEAFAKRQGKEMDSL | |
| | TFLYDGIEIQADQTPEDLDMEDNDI | |
| | IEAHREQIGG | |
| aMTD524-P2- | AVALIVVPALAPEAAAKLIRDSSDQ | 257 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD343-P2- | IVAVALPALVAPEAAAKLIRDSSDQ | 258 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD385-P2- | IVAIAVPALVAPEAAAKLIRDSSDQ | 259 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD485-P2- | AILAAIVPLAVPEAAAKLIRDSSDQ | 260 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD830-P2- | IALVAAPVALVPEAAAKLIRDSSDQ | 261 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD524-P2- | AVALIVVPALAPEAAAKLIRDSSDQ | 262 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD343-P2- | IVAVALPALVAPEAAAKLIRDSSDQ | 263 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD385-P2- | IVAIAVPALVAPEAAAKLIRDSSDQ | 264 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD485-P2- | AILAAIVPLAVPEAAAKLIRDSSDQ | 265 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD830-P2- | IALVAAPVALVPEAAAKLIRDSSDQ | 266 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |

Moreover, the present disclosure provides not only the amino acid sequence of the CP-SD recombinant protein, but also a polynucleotide encoding the same, a recombinant expression vector comprising the polynucleotide, and a transformant transformed with the recombinant expression vector.

III. Use of CP-SD Recombinant Protein

1. Use for Binding to Leptin Receptor

The present disclosure provides a CP-SD recombinant protein binding to a leptin receptor. The CP-SD recombinant protein provided according to the present disclosure can be used in binding to a leptin receptor because a partial region of SH2 domain capable of binding to a leptin receptor is included therein.

Furthermore, the CP-SD recombinant protein binding to a leptin receptor can be used to inhibit the binding of a substance binding to the leptin receptor according to an embodiment. In an exemplary embodiment, the CP-SD recombinant protein may inhibit the binding of SOCS3, which binds to a leptin receptor. In addition, this inhibition may lead to preventing the degradation of a leptin receptor, which is induced by SOCS3 binding.

Additionally, the CP-SD recombinant protein provided according to the present disclosure can be more effectively delivered to plasma membranes and bind to the leptin receptor because the recombinant protein comprises an aMTD domain which aids penetration to plasma membranes of cells.

Moreover, the recombinant protein can inhibit the binding of leptin receptor-binding SOCS3, thereby suppressing the leptin receptor degradation induced by the binding of SOCS3.

2.Pharmaceutical Composition

The present disclosure provides a composition comprising the CP-SD recombinant protein. The present disclosure provides a composition comprising the CP-SD recombinant protein as an active ingredient. In one embodiment, the composition may be a pharmaceutical composition for treatment or prevention of a disease.

The pharmaceutical composition provided according to the present disclosure may further comprise a vehicle. The pharmaceutically acceptable vehicle contained in the pharmaceutical composition of the present disclosure is usually used for formulation. Examples of the vehicle include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxy benzoate, propyl hydroxy benzoate, talc, magnesium stearate, mineral oil, and the like, but are not limited thereto. In addition to the above ingredients, the pharmaceutical composition of the present disclosure may further contain a lubricant, a wetting agent, sweetener, a colorant, a flavorant, an emulsifier, a suspending agent, a preservative, and the like. For details of pharmaceutically acceptable vehicles and suitable formulations, reference may made to Remington's Pharmaceutical Sciences (19th ed., 1995).

The pharmaceutical composition according to the present disclosure may be formulated using at least one diluent or excipient, usually used in the art, such as a filler, an extender, a binder, a wetting agent, a disintegrant, a surfactant, and so on.

In one embodiment, solid formulations for oral administration include tablets, pills, powders, granules, capsules, troches, etc. These solid formulations may be prepared by mixing at least one compound of the present disclosure with one or more excipients, for example, starch, calcium carbonate, sucrose, lactose, gelatin, etc. In addition, a lubricant such as magnesium stearate, talc, etc. is employed in addition to simple excipients. In another embodiment, liquid formulations for oral administration include a suspension, a solution for internal use, an emulsion, a syrup, etc. In addition to water commonly used as a simple diluent and liquid paraffin, various excipients, for example, wetting agents, sweetening agents, flavors, preservatives, etc. may be included. Formulations for parenteral administration include sterilized aqueous solutions, non-aqueous solvents, suspending agents, emulsions, lyophilizates, suppositories, etc. Propylene glycol, polyethylene glycol, vegetable oils such as olive oil, injectable esters such as ethyl oleate, etc. may be used as non-aqueous solvents and suspending agents. Bases for suppositories may include witepsol, macrogol, tween 61, cacao butter, laurin butter, glycerinated gelatin, etc.

Competing for binding to a leptin receptor with SOCS3, the CP-SD recombinant protein can inhibit the binding of SOCS3 to the leptin receptor. That is, the leptin receptor-binding peptide can be contained in a pharmaceutical composition for prevention, treatment, or alleviation of a disease caused by the binding of SOCS3 to a leptin receptor or can be used as a medicine or for preparing a medicine. In one embodiment, the disease caused by binding of SOCS3 to a leptin receptor includes obesity, especially, leptin-resistant obesity. Furthermore, the disease may be an obesity-related diseases, examples of which include depression, intracranial hypertension, dementia, heart attack, vascular sclerosis, irregular menstruation, cancer, arthritis, asthma, fatty liver, diabetes mellitus, hyperlipidemia, high blood pressure, gallbladder disease, coronary artery disease, gout, and stroke, but are not limited thereto. Any disease that is known as an obesity-related disease may be included.

3.Method of Treating

The CP-SD recombinant protein of the present disclosure can be used for treating a disease. More specifically, the present disclosure provides a method for treatment of a disease, the method comprising administering a composition comprising the CP-SD recombinant protein to a subject in need thereof. In this context, the subject may mean a mammal including humans.

According to intended modalities, the composition provided in the present disclosure may be orally or parenterally administered (for example, intravenously, subcutaneously, intraperitoneally, or topically). Administration doses may be properly determined by a person skilled in the art, depending on patient's state and body weight, the severity of disease, dosage forms of drugs, administration routes and time, etc.

The composition according to the present disclosure is administered in a pharmaceutically effective amount. As used herein, the term “pharmaceutically effective amount” refers to an amount sufficient to treat diseases, at a reasonable benefit/risk ratio applicable to any medical treatment. The effective dosage level may be determined depending on various factors including the type and severity of disease, the activity of drugs, the sensitivity to drugs, the time of administration, the route of administration, excretion rate, the duration of treatment, drugs used in combination with the composition, and other factors known in the medical field. The composition of the present invention may be administered as a sole therapeutic agent or in combination with other therapeutic agents, and may be administered sequentially or simultaneously with conventional therapeutic agents. The composition can be administered in a single or multiple dosage form. It is important to administer the composition in the minimum amount that can exhibit the maximum effect without causing side effects, in view of all the above-described factors, and the amount can be easily determined by a person skilled in the art.

In detail, an effective amount of the compound according to the present disclosure may vary depending on the age, sex, and body weight of the patient. Generally, the compound may be administered in an amount of 0.1 to 100 mg per kg of body weight and preferably in an amount of 0.5 to 10 mg per kg of body weight every day or every other day, or one to three times a day. The dose may be increased or decreased depending on administration route, severity of obesity, sex, body weight, age, etc. and thus does not limit the scope of the present disclosure in any way.

In the context of the method for treatment of a disease, the disease includes all the disclosure of section 2. Pharmaceutical composition on diseases. That is, the present disclosure provides a method for treatment of leptin-resistant obesity, the method comprising administering a pharmaceutical composition comprising the CP-SD recombinant protein to a subject in need thereof. In addition, the present invention provides a method for treatment of obesity-related diseases including depression, intracranial hypertension, dementia, heart attack, vascular sclerosis, irregular menstruation, cancer, arthritis, asthma, fatty liver, diabetes mellitus, hyperlipidemia, high blood pressure, gallbladder disease, coronary artery disease, gout, and stroke, the method comprising administering a pharmaceutical composition comprising the CP-SD recombinant protein to a subject in need thereof.

Hereinafter, the present disclosure will be described in further detail with reference to the following examples. It is to be understood, however, that these examples are for illustrative purposes only and are not intended to limit the scope of the present invention.

### Examples

1. Cloning of Cell-Permeable Truncated SOCS3 SH2 Domain Recombinant Proteins

Full-length cDNA for human SOCS3 was purchased from Origene (USA). The truncated SOCS3 SH2 domain proteins were constructed by amplifying some of the specific regions of the SOCS3 ORF (225 amino acids) in the amino acid sequence of the SH2 domain from G45 to N185 [SH2 (L69-Q96(LIRDSSDQRHFFTLSVKTQSGTKNLRIQ(SEQ ID No:1))), SH2 (V120-M128(VLKLVHHYM(SEQ ID No:2))), SH2 (H125-M128(HHYM(SEQ ID No:3))) and SH2 (A164-N185(AYYIYSGGEKIPLVLSRPLSSN(SEQ ID No:4)))] using primers for insertion of linkers at the ends of the specific SH2 domain regions and for fusion of aMTD/SD to truncated SOCS3 SH2 domain proteins. The aMTD-fused or aMTD/SD-fused truncated SOCS3 SH2 domain proteins were subcloned into a 6×His-tagged pET-28a(+) or tag free pET-26b(+) expression vector (Merck Millipore). The resulting plasmids were sequenced.

2. Preparation of Cell-Permeable Truncated SOCS3 SH2 Domain Recombinant Proteins

Recombinant vectors encoding P2-1-6, P2-1-21, P2-1-23, P2-1-24, 36, 37 or P2-1-32B were transformed into E. coli strains [BL21 (DE3)-CodonPlus-RIL, BL21 (DE3)-CodonPlus-RIPL, TUNER (DE3), Rosetta [symbol] (DE3), BL21-Gold (DE3)] using heat shock method. Next, E. coli containing the vectors was cultured in kanamycin-containing LB broth at 37° C. for over-night, and transferred into 5 L kanamycin-containing LB broth and cultured using fermenter at 37° C. until the OD₆₀₀ reached the value of 0.8 or 1.2. Then, 0.7 mM isopropyl-β-D-thiogalactoside (IPTG, Gen Depot, USA) was added into the fermenter and processed additional 3 hours culture at 37° C.

E. coli containing P2-1B, P2-1E or P2-1-24B recombinant protein sequence was cultured in kanamycin containing LB broth at 37° C. for over-night, and transferred into 5 L kanamycin-containing LB broth and cultured using fermenter at 37° C. until the OD₆₀₀ reached the value of 0.6-0.8. Then, 0.3 mM of isopropyl-β-D-thiogalactoside (IPTG, Gen Depot, USA) was added into the fermenter and processed additional 16 hours culture at 25° C.

The culture medium was centrifuged at 4° C. and 8,000 rpm for 10 minutes and a supernatant was discarded to recover the cell pellet. The strain lysate where protein expression was not induced and the strain lysate where protein expression was induced by addition of IPTG were loaded on SDS-PAGE to analyze protein expression levels.

The purification of aMTD524-P2-1-6, aMTD524-P2-1-21, aMTD524-P2-1-23, aMTD524-P2-1-24, aMTD524-36 and aMTD524-37 was performed using following methods. The cell pellet thus recovered was suspended in a lysis buffer (50 mM HEPES, 100 mM NaCl, pH 7.5), and cells were disrupted by sonication (on/off time: 5 sec/10 sec, on time 30 mins, amplify 60%), and centrifuged at 4° C. and 8,000 rpm for 45 min to obtain a soluble fraction and an insoluble fraction. This insoluble fraction was suspended in a denature buffer (8 M urea, 50 mM HEPES, 100 mM NaCl, 20 mM imidazole, pH 7.5). aMTD524-P2-1-6 was purified by Ni²⁺ affinity chromatography with a binding buffer (8 M urea, 50 mM HEPES, 100 mM NaCl, 20 mM imidazole, pH 7.5) and a elution buffer (8 M urea, 50 mM HEPES, 100 mM NaCl, 500 mM imidazole, pH 7.5). aMTD524-P2-1-21, aMTD524-P2-1-23 and aMTD524-P2-1-24 were purified by cation exchange chromatography by Hi Trap SP-HP (GE Healthcare) with a binding buffer (8 M urea, 50 mM sodium acetate, pH 4.0) and an elution buffer (8 M urea, 50 mM sodium acetate, 1 M NaCl, pH 4.0). The eluted proteins were refolded by dilution in a refolding buffer (50 mM sodium acetate, 250 mM NaCl, 400 mM L-Arg, 2 mM EDTA, 5% sorbitol, pH 4.0) and incubation for 20 hr, and were purified by size exclusion chromatography by Hi Load 26/60 superdex 200 pg (GE Healthcare) with a storage buffer (50 mM sodium acetate, 50 mM NaCl, pH 4.0).

The purification of aMTD524-P2-1B, aMTD524-P2-1-24B and aMTD524-P2-1-32B was performed using the following methods. The cell pellet thus recovered was suspended in a lysis buffer (50 mM Tris, 50 mM NaCl, pH 7.5), and cells were disrupted by sonication (on/off time: 5 sec/10 sec, on time 30 mins, amplify 60%), and centrifuged at 4° C. and 8,000 rpm for 45 min to obtain a soluble fraction and an insoluble fraction. This soluble fraction was diluted in an elution buffer for anion exchange chromatography and was purified by anion exchange chromatography by Q-sepharose-HP (GE Healthcare) with a binding buffer (50 mM Tris, pH 7.5) and an elution buffer (50 mM Tris, 1 M NaCl, pH 7.5). The eluted proteins were purified by hydrophobic interaction chromatography by HiTrap Butyl HP (Cytiva) with a binding buffer (50 mM Tris, 1.5 M NaCl, pH 7.5) and an elution buffer (50 mM Tris, pH 7.5). The eluted proteins were purified by size exclusion chromatography by Hi Load 26/60 superdex 200 pg (GE Healthcare) with a storage buffer (50 mM Tris, 150 mM NaCl, pH 7.5).

aMTD-replaced CP-SD recombinant proteins, aMTD830-P2-1B, aMTD830-P2-1-24B and aMTD830-P2-1-32B, were purified using the same protocol as the aMTD524-fused CP-SD recombinant proteins.

The purified proteins were loaded on SDS-PAGE gel and analyzed by SE-HPLC to analyze protein expression characteristics.

aMTD524-P8, aMTD524-P8-1, aMTD524-P9, aMTD524-P10, aMTD524-P10-1, aMTD524-P11, aMTD524-P11-1, aMTD524-P12, aMTD524-P13 and aMTD524-P14 was acquired by peptide synthesis from Anygen (Gwangju, Korea).

3. Testing cell-permeability of cell-permeable truncated SOCS3 SH2 domain recombinant proteins

To test cell-permeability of aMTD-fused truncated SOCS3 SH2 domain (CP-SD) recombinant proteins, FITC-aMTD524-P2-1, FITC-conjugated aMTD524-P2-1 [AVALIVVPALAPEAAAKLIRDSSDQRHFFTLSVK(FITC)TQSGTKNLRIQ], was acquired by peptide synthesis from Anygen (Gwangju, Korea). RAW 264.7 (Korean Cell Line Bank, Seoul, Korea) was maintained in DMEM media containing 10% fetal bovine serum (FBS).

FITC-aMTD524-P2-1 was diluted into serum free DMEM at concentration of 20 μM and added to RAW 264.7 cells for 3 hours, washed with ice-cold PBS for at least 5 times and trypsin-EDTA was treated for 3 mins, and then washed with ice-cold PBS for at least 3 times to remove any proteins which did not penetrate into cells. FITC-SH2 (L69-Q96) [LIRDSSDQRHFFTLSVK(FITC)TQSGTKNLRIQ], the structure which does not contain aMTD, and unconjugated FITC were added to the cells using the same protocol as the control groups. Next, the cells were fixed using 70% ethanol at −20° C. for 15 minutes, and analysed using FACS (BD FACS LSR II SORP system, Becton Dickinson company). Also, the same cells used for flow cytometry analysis were mounted on slide glass using DAPI (4′,6-diamidino-2-phenylindole) added mounting medium, and visualized using confocal laser microscope (LSM700, Zeiss, Germany).

4. Binding Affinity of Cell-Permeable Truncated SOCS3 SH2 Domain Recombinant Proteins to Leptin Receptor

The binding affinity of cell-permeable truncated SOCS3 SH2 domain recombinant proteins to leptin receptor was analysed using isothermal titration calorimetry (ITC). Phosphorylated human leptin receptor peptide (QRQPFVK[pY] ATLISNSK) used in the experiment was acquired from Anygen (Gwangju, Korea). The binding affinity of aMTD524-P2-1-6 was measured using the following methods. Leptin receptor peptide was dissolved into DMSO and was diluted into storage buffer (50 mM sodium acetate, 50 mM NaCl, pH 4.0) to reach a final peptide concentration of 400 μM and final DMSO concentration of 0.5%. Using dialysis method, purified aMTD524-P2-1-6 was maintained at the identical batch storage buffer, and was diluted to reach the final concentration of 50 μM. Next, binding affinity was measured using ITC (Auto-ITC200).

5. Biological Activity Test of Cell-Permeable Truncated SOCS3 SH2 Domain Recombinant Proteins

To validate that cell-permeable truncated SOCS3 recombinant protein can overcome leptin resistance, the purified recombinant proteins were treated at leptin resistant cell line mHypoA2/21-SOCS3. mHypoA2/21-SOCS3 was produced by over-expressing SOCS3 at mouse hypothalamic neuron mHypoA2/21, and cultured using DMEM containing 10% FBS and 7 mg/mL geneticin. Cells were seeded at the concentration of 150000 cells/60 mm plate and on the following day the medium was replaced with 7 mg/mL geneticin containing serum free DMEM and starved the cells for the following 16 hours. Purified CP-SD recombinant proteins were diluted into 7 mg/mL geneticin containing serum free DMEM and treated for 1 hour, proteins were removed, and 10 ng/mL mouse leptin was treated for 30 minutes.

The cells were lysed in a lysis buffer (150 mM NaCl, 20 mM Tris, 1% triton-X-100, pH 7.5) containing protease inhibitor and proteinase inhibitor, incubated for 20 min at 4° C., and centrifuged at 13,000 rpm for 20 min at 4° C. Equal amounts of lysates were separated on 10% SDS-PAGE gels and transferred to a PVDF membrane. The membranes were blocked using TBST buffer 5% bovine serum albumin (BSA) and for western blot analysis incubated with the following antibodies diluted in TBST buffer 5% bovine serum albumin (BSA): anti-phospho-STAT3 (Cell Signaling Technology, USA), anti-STAT3 (Cell Signaling Technology, USA) and anti-myc (Cell Signaling Technology, USA), then HRP conjugated anti-rabbit secondary antibody (Cell Signaling Technology, USA) and anti-mouse secondary antibody (Cell Signaling Technology, USA). The protein bands were detected with ECL solution using luminescent image analyzer (ImageQuant LAS 500, GE Healthcare).

6. Turbidity of Cell-Permeable Truncated SOCS3 SH2 Domain Recombinant Proteins with Thermal Stress

The recombinant proteins were diluted in their storage buffer or serum free DMEM medium and added into wells of a clear 96 well plate. The plate was incubated at 37° C. and the absorbance of each well was measured at 350 nm every 30 minutes in a microplate reader (Synergy H1, BioTek).

### Experimental Examples

1. The Core Sequence of Cell-Permeable Truncated SOCS3 SH2 Domain (CP-SD) Recombinant Proteins to Overcome Leptin Resistance

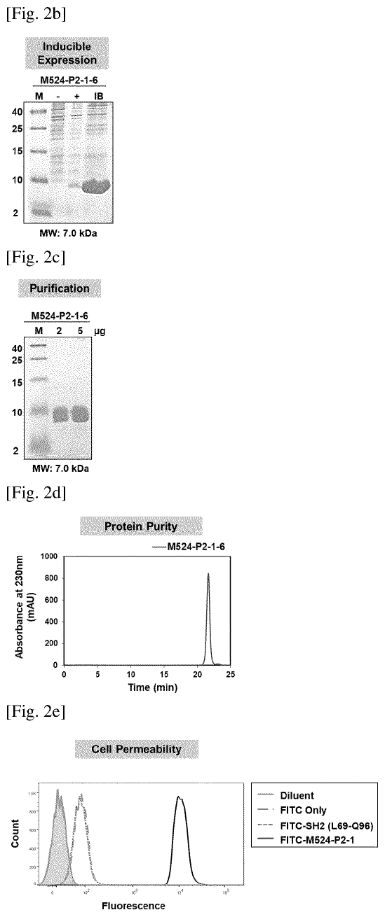

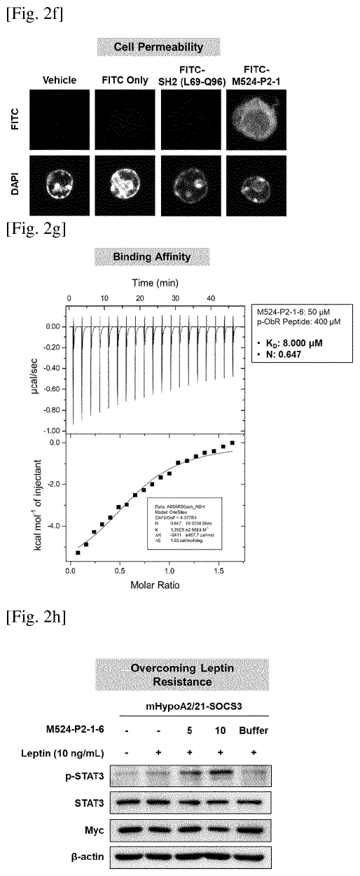

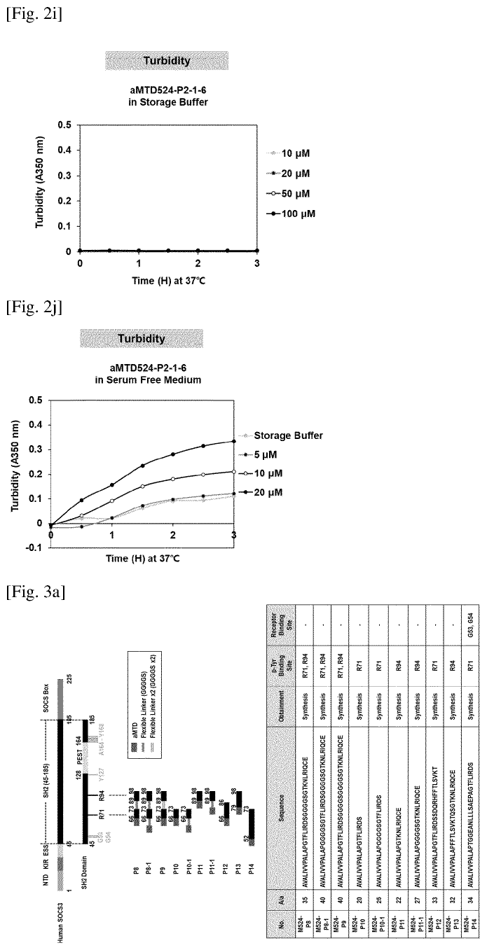

The SH2 domain of SOCS3 has two residues, R71 and R94, for binding to the phosphorylated Y985 of the leptin receptor. To demonstrate whether a truncate including R71 and R94 binds to the activated leptin receptor, aMTD524-P2-1 which consists of aMTD524(AVALIVVPALAP(SEQ ID NO:126)) and SH2 (L69-Q96(SEQ ID NO:1)), was designed. To purify aMTD524-P2-1, his tag-conjugated aMTD524-P2-1, aMTD524-P2-1-6, was designed (FIG. 2a).

TABLE 3
Designed P2 Sequence

| | | SEQ |
|---|---|---|
| | | ID |
| No. | Sequence | No. |
| aMTD524- | AVALIVVPALAPEAAAKLIRDS | 267 |
| P2-1 | SDQRHFFTLSVKTQSGTKNLRI | |
| | Q | |
| aMTD524- | GSSHHHHHHSSGLVPRGSHMAV | 268 |
| P2-1-6 | ALIVVPALAPEAAAKLIRDSSD | |
| | QRHFFTLSVKTQSGTKNLRIQ | |

aMTD524-P2-1-6 was expressed in E. coli (FIG. 2b). The homogeneity of aMTD524-P2-1-6 was validated through single band in SDS-PAGE, and single peak in SE-HPLC analysis (FIGS. 2c, d). To determine cell-permeability, FITC-labeled aMTD524-P2-1 was treated in cells and cell uptake was determined. The flow cytometry analysis result showed the cell permeability of FITC-aMTD524-P2-1 was 100.8 times better than FITC-labeled SH2 (L69-Q96) (FIG. 2e). The fluorescence intensity of FITC-aMTD524-P2-1 treated cells was strong compared to the FITC-SH2 (L69-Q96) and unconjugated FITC treated cells (FIG. 2f).

The purified aMTD524-P2-1-6 binds to the leptin peptide phosphorylated at Y985 with the binding affinity of KD=8.0 μM (FIG. 2g). Whether the purified aMTD524-P2-1-6 can induce leptin-mediated activation of JAK/STAT signaling was verified. While leptin did not induce STAT3 phosphorylation in leptin resistant mHypoA2/21-SOCS3 cells, aMTD524-P2-1-6 overcame leptin resistance with increase in STAT3 phosphorylation level (FIG. 2h). aMTD524-P2-1-6 was stable with thermal stress of 37° C. for 3 hours in the storage buffer but not in serum free medium (FIG. 2i, j).

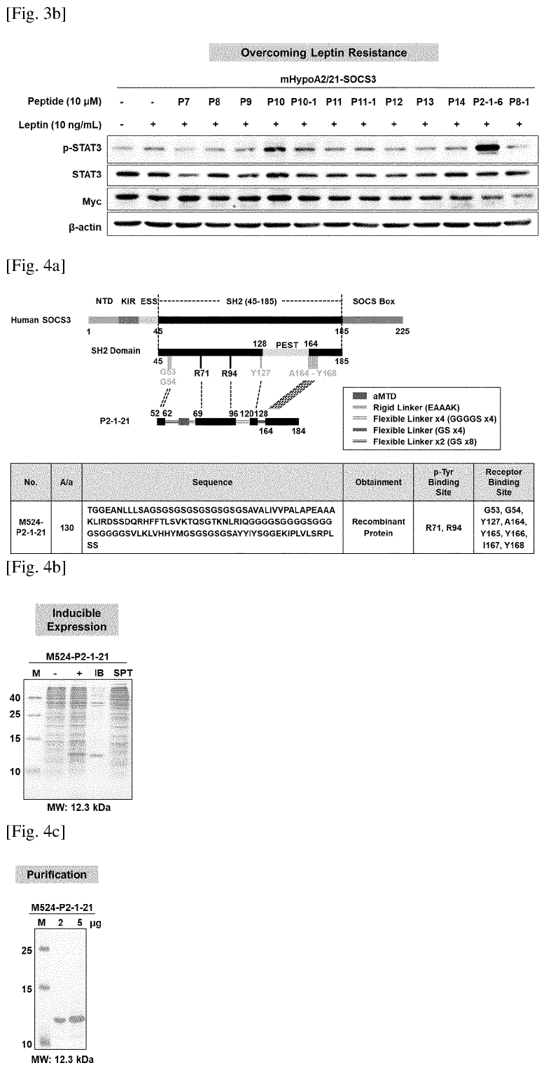

Other truncates including R71 and R94 were also designed (FIG. 3a). aMTD524-P8, aMTD524-P8-1 and aMTD524-P9 include two of the three strands which consist of the full β-sheet structure (G66-E98). In addition, truncates including one of the p-Tyr binding sites were designed. aMTD524-P10, aMTD524-P10-1, aMTD524-P12 and aMTD524-P13 include R71 in one or two strands of the β-sheet (G66-E98). aMTD524-P11 and aMTD524-P11-1 include R94 in a strand of the β-sheet (G66-E98). aMTD524-P14 includes a strand of the β-sheet (G66-E98) with R71 and an α-helical region with G53 and G54.

TABLE 4
Designed P8 to P14 Sequences

| | | SEQ |
|---|---|---|
| | | ID |
| No. | Sequence | No. |
| aMTD524-P8 | AVALIVVPALAPGTFLIRDSG | 269 |
| | GGGSGTKNLRIQCE | |
| aMTD524- | AVALIVVPALAPGGGGSGTFLI | 270 |
| P8-1 | RDSGGGGSGTKNLRIQCE | |
| aMTD524-P9 | AVALIVVPALAPGTFLIRDSG | 271 |
| | GGGSGGGGGSGTKNLRIQCE | |
| aMTD524-P10 | AVALIVVPALAPGTFLIRDS | 272 |
| aMTD524- | AVALIVVPALAPGGGGSGTFL | 273 |
| P10-1 | IRDS | |
| aMTD524-P11 | AVALIVVPALAPGTKNLRIQCE | 274 |
| aMTD524- | AVALIVVPALAPGGGGSGTKNL | 275 |
| P11-1 | RIQCE | |
| aMTD524-P12 | AVALIVVPALAPGTFLIRDSSD | 276 |
| | QRHFFTLSVKT | |
| aMTD524-P13 | AVALIVVPALAPFFTLSVKTQS | 277 |
| | GTKNLRIQCE | |
| aMTD524-P14 | AVALIVVPALAPTGGEANLLLS | 278 |
| | AEPAGTFLIRDS | |

However, none of the 11 structures overcame leptin resistance (FIG. 3b).

These results demonstrated that aMTD524-P2-1-6, his tag-fused aMTD524-P2-1, is soluble, homogeneous and cell-permeable proteins and it binds to the activated leptin receptor and overcomes leptin resistance. It implies that SH2 (L69-Q96) is enough to bind to the receptor and competes with endogenous SOCS3 and that structures which partially include SH2 (L69-Q96) lose the function of SH2 (L69-Q96). Based on these results, other CP-SD recombinant proteins were designed, which contains SH2 (L69-Q96) as the core sequence of CP-SD.

2. Cell-Permeable Truncated SOCS3 SH2 Domain (CP-SD) Recombinant Proteins with the Core Sequence and Additional Receptor Binding Residues

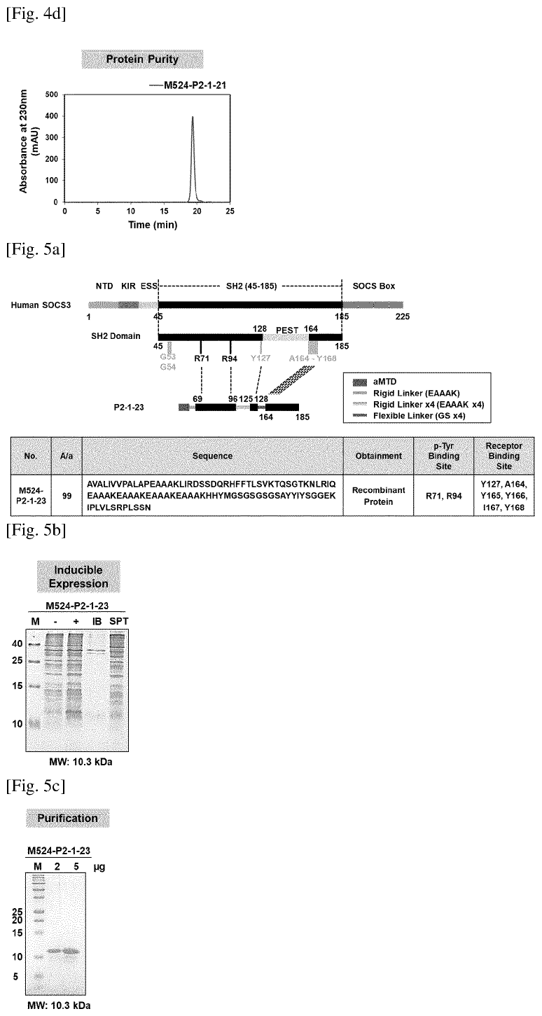

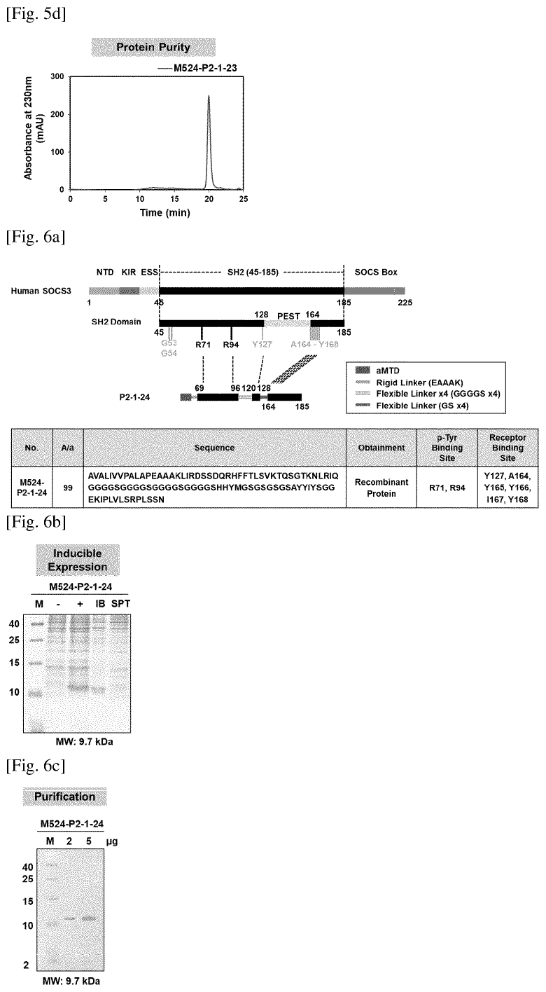

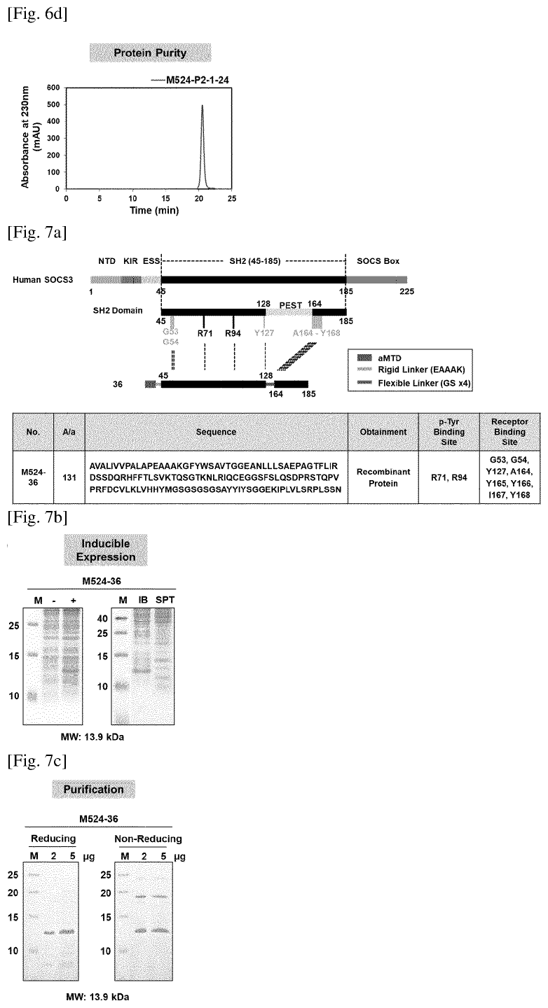

CP-SD recombinant proteins were designed, which consist of aMTD, SH2 (L69-Q96) and additional receptor binding residues in the SH2 domain without a his tag. The three CP-SD recombinant proteins, aMTD524-P2-1-21 containing SH2 (T52-A62), H2 (L69-Q96), SH2 (V120-M128) and SH2 (A164-N185), aMTD524-P2-1-23 containing SH2 (L69-Q96), SH2 (H125-M128) and 2H2 (A164-N185) and aMTD524-P2-1-24 containing the same regions as aMTD524-P2-1-23 with different linkers, were expressed in E. coli and successfully purified from the inclusion body (FIGS. 4a-b, 5a-b and 6a-b).

TABLE 5
P2-1 Sequences with Different Linkers

| | | SEQ |
|---|---|---|
| | | ID |
| No. | Sequence | No. |
| aMTD- | TGGEANLLLSAGSGSGSGSGSGSGSGSAVALIVV | 279 |
| P2-1-21 | PALAPEAAAKLIRDSSDQRHFFTLSVKTQSGTKN | |
| | LRIQGGGGSGGGGSGGGGSGGGGSVLKLVHHY | |
| | MGSGSGSGSAYYIYSGGEKIPLVLSRPLSS | |
| aMTD- | AVALIVVPALAPEAAAKLIRDSSDQRHFFTLSVK | 280 |
| P2-1-23 | TQSGTKNLRIQEAAAKEAAAKEAAAKEAAAKH | |
| | HYMGSGSGSGSAYYIYSGGEKIPLVLSRPLSSN | |
| aMTD-4 | AVALIVVPALAPEAAAKLIRDSSDQRHFFTLSVK | 281 |
| P2-1-2 | TQSGTKNLRIQGGGGSGGGGSGGGGSGGGGSH | |
| | HYMGSGSGSGSAYYIYSGGEKIPLVLSRPLSSN | |

The purified aMTD524-P2-1-21, aMTD524-P2-1-23 and aMTD524-P2-1-24 were homogenous with single band in SDS-PAGE and a single peak in SE-HPLC analysis (FIGS. 4c-d, 5c-d and 6c-d). aMTD524-P2-1-24 overcame leptin resistance, but aMTD524-P2-1-21 and aMTD524-P2-1-23 did not (FIGS. 9a, b).

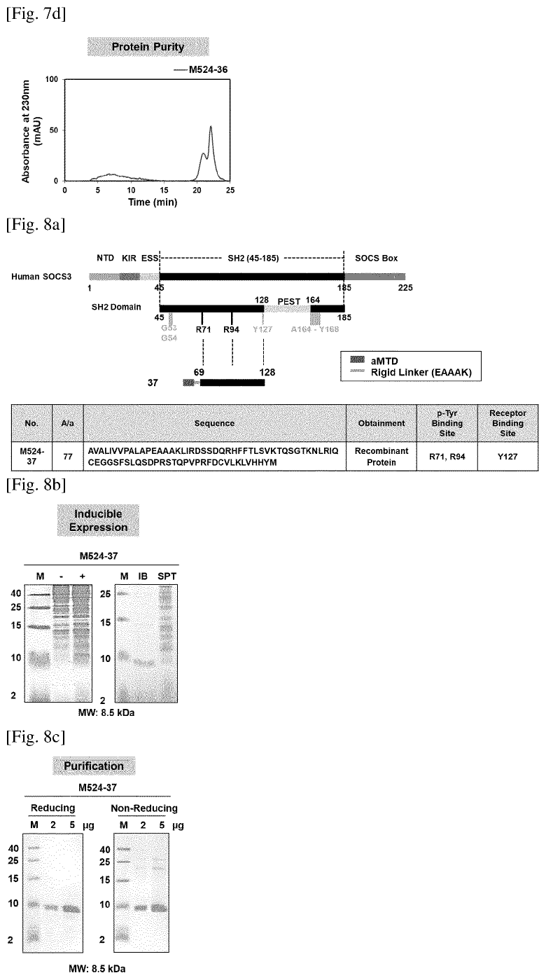

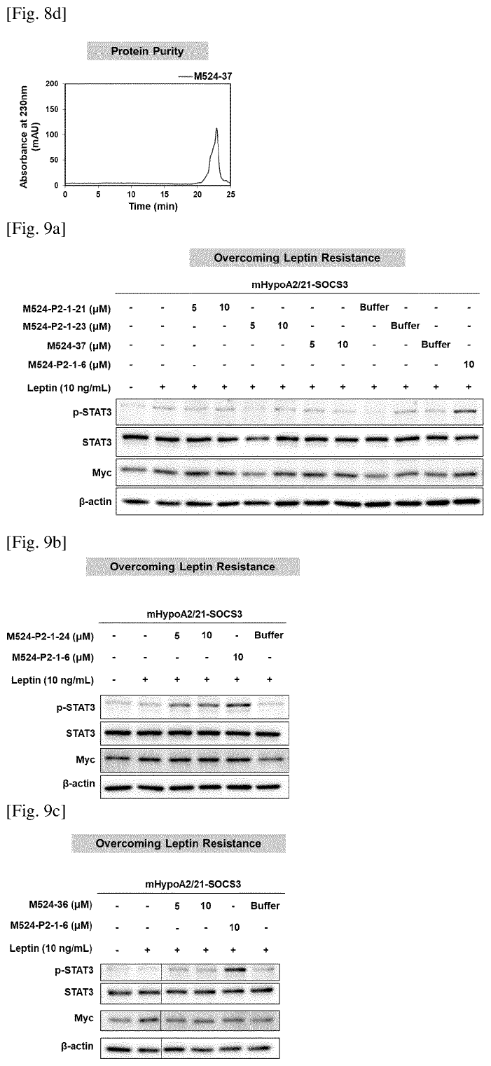

Other truncates from the SH2 domain, aMTD524-36 and aMTD524-37, were designed, which contain aMTD and SH2 (L69-Q96) but do not follow the first systemic approach for CP-SD recombinant proteins (FIGS. 7a and 8a).

TABLE 6
Designed Sequences 36 to 37

| | | SEQ |
|---|---|---|
| | | ID |
| No. | Sequence | No. |
| aMTD524- | AVALIVVPALAPEAAAKGFYWSAVTGGEANLLL | 282 |
| 36 | SAEPAGTFLIRDSSDQRHFFTLSVKTQSGTKNL | |
| | RIQCEGGSFSLQSDPRSTQPVPRFDCVLKLVHH | |
| | SYMGGSGSGSAYYIYSGGEKIPLVLSRPLSSN | |
| aMTD524- | AVALIVVPALAPEAAAKLIRDSSDQRHFFTLSV | \|283 |
| 37 | QKTSGTKNLRIQCEGGSFSLQSDPRSTQPVPRF | |
| | DCVLKLVHHYM | |

aMTD524-36 and aMTD524-37 were expressed in E. coli and purified from the inclusion body (FIGS. 7b and 8b). However, these two purified recombinant proteins were heterogeneous and did not overcome leptin resistance (FIGS. 7c-d, 8c-d and 9a, c).

3. Cell-Permeable Truncated SOCS3 SH2 Domain (CP-SD) Recombinant Proteins with the Core Sequence, Additional Receptor Binding Residues and a Solubilization Domain (SD)

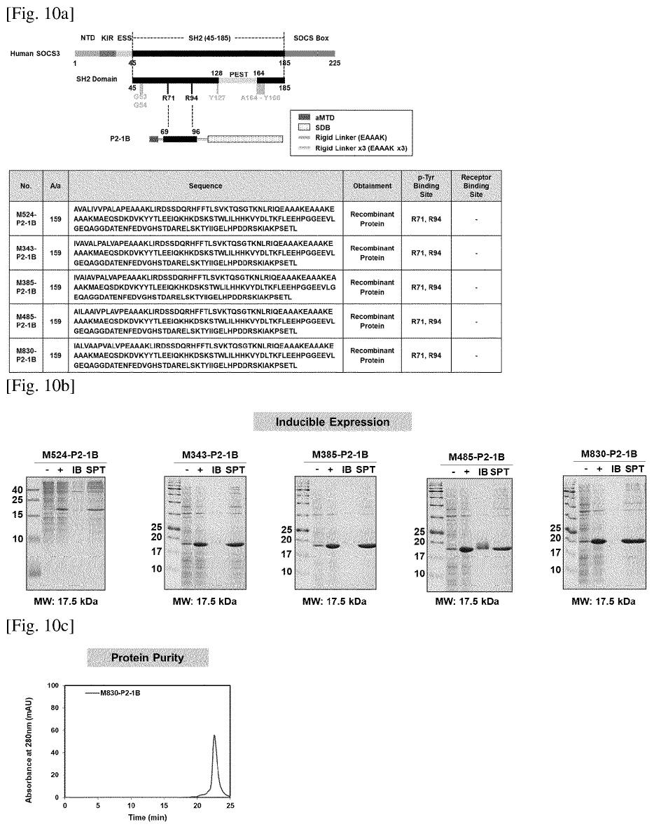

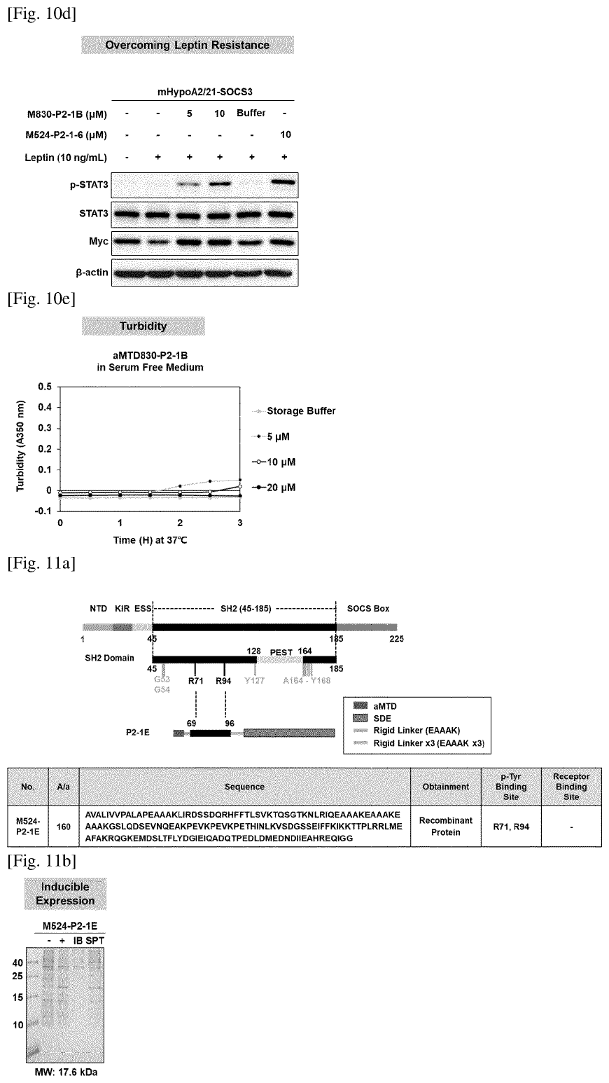

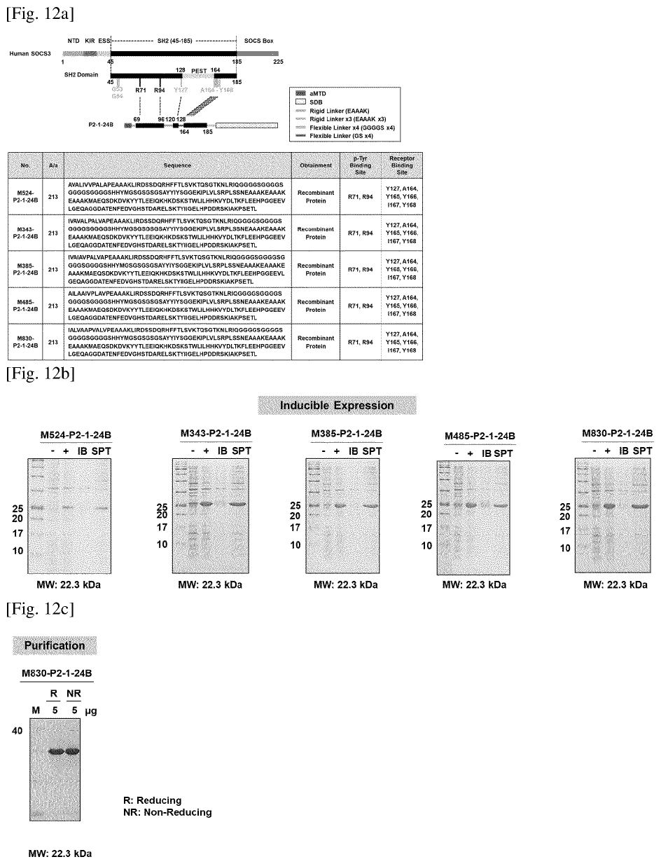

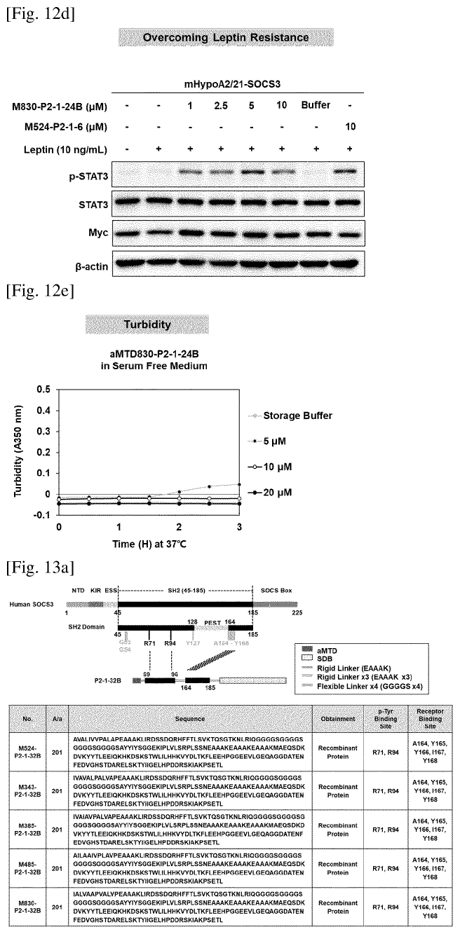

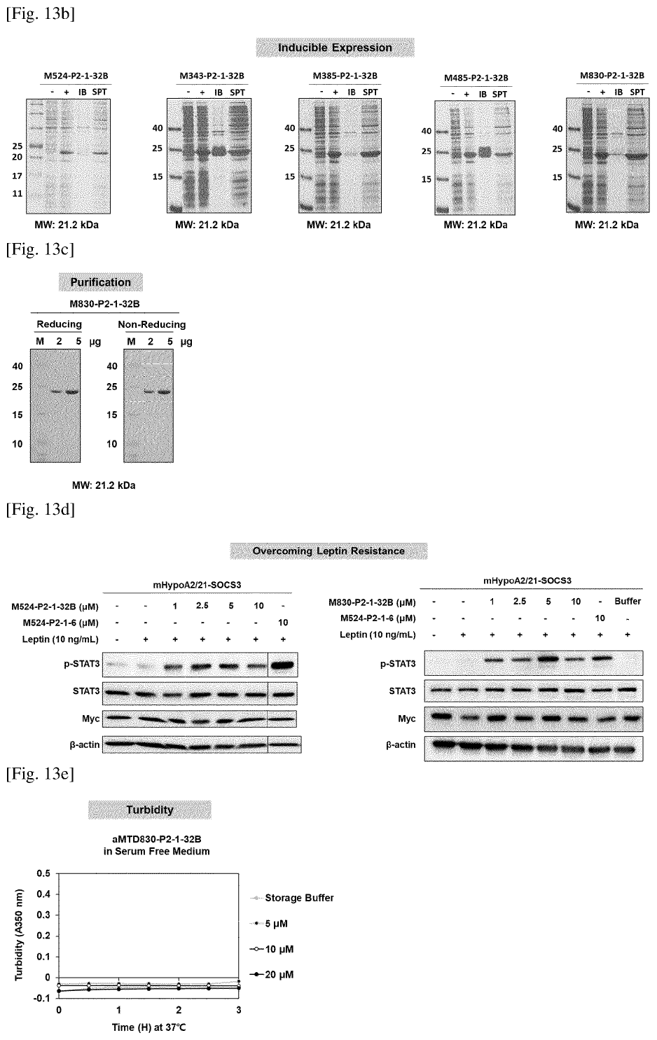

To improve solubility of purified proteins, solubilization domains (SDs)-fused CP-SD recombinant proteins were additionally designed; aMTD524-P2-1B containing SH2 (L69-Q96) and SDB, aMTD524-P2-1-E containing SH2 (L69-Q96) and SDE, aMTD524-P2-1-24B containing SH2 (L69-Q96), SH2 (H125-M128), SH2 (A164-N185) and SDB, aMTD524-P2-1-32B containing SH2 (L69-Q96), SH2 (A164-N185) and SDB (FIGS. 10a, 11a, 12a and 13a). The four CP-SD recombinant proteins were expressed in E. coli and the conjugation of SD induced the existence of the expressed proteins mainly in the supernatant after cell lysis instead of the inclusion body (FIGS. 10b, 11b, 12b and 13b).

TABLE 7
Sequence comprising Solubilization
domain(SD)

| | | SEQ |
|---|---|---|
| | | ID |
| No. | Sequence | No. |
| aMTD524-P2- | AVALIVVPALAPEAAAKLIRDSSDQ | 251 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD343-P2- | IVAVALPALVAPEAAAKLIRDSSDQ | 252 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD385-P2- | IVAIAVPALVAPEAAAKLIRDSSDQ | 253 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD485-P2- | AILAAIVPLAVPEAAAKLIRDSSDQ | 254 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD830-P2- | IALVAAPVALVPEAAAKLIRDSSDQ | 255 |
| 1B | RHFFTLSVKTQSGTKNLRIQEAAAK | |
| | EAAAKEAAAKMAEQSDKDVKYYTLE | |
| | EIQKHKDSKSTWLILHHKVYDLTKF | |
| | LEEHPGGEEVLGEQAGGDATENFED | |
| | VGHSTDARELSKTYIIGELHPDDRS | |
| | KIAKPSETL | |
| aMTD524-P2- | AVALIVVPALAPEAAAKLIRDSSDQ | 256 |
| 1E | RHFFTLSVKTQSGTKNLRIQEAAAK | |
| | EAAAKEAAAKGSLQDSEVNQEAKPE | |
| | VKPEVKPETHINLKVSDGSSEIFFK | |
| | IKKTTPLRRLMEAFAKRQGKEMDSL | |
| | TFLYDGIEIQADQTPEDLDMEDNDI | |
| | IEAHREQIGG | |
| aMTD524-P2- | AVALIVVPALAPEAAAKLIRDSSDQ | 257 |
| 1-24B | RHFFTLSVKTQSGTKNLRIQGGGGS | |
| | GGGGSGGGGSGGGGSHHYMG | |
| | SGSGSGSAYYIYSGGEKIPLVLSRP | |
| | LSSNEAAAKEAAAKEAAAKMAEQSD | |
| | KDVKYYTLEEIQKHKDSKSTWLILH | |
| | HKVYDLTKFLEEHPGGEEVLGEQAG | |
| | GDATENFEDVGHSTDARELSKTYII | |
| | GELHPDDRSKIAKPSETL | |
| aMTD343-P2- | IVAVALPALVAPEAAAKLIRDSSDQ | 258 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD385-P2- | IVAIAVPALVAPEAAAKLIRDSSDQ | 259 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD485-P2- | AILAAIVPLAVPEAAAKLIRDSSDQ | 260 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD830-P2- | IALVAAPVALVPEAAAKLIRDSSDQ | 261 |
| 1-24B | RHFFTLSVKTQSGTKNLRIQGGGGS | |
| | GGGGSGGGGSGGGGSHHYMGSGSGS | |
| | GSAYYIYSGGEKIPLVLSRPLSSNE | |
| | AAAKEAAAKEAAAKMAEQSDKDVKY | |
| | YTLEEIQKHKDSKSTWLILHHKVYD | |
| | LTKFLEEHPGGEEVLGEQAGGDATE | |
| | NFEDVGHSTDARELSKTYIIGELHP | |
| | DDRSKIAKPSETL | |
| aMTD524-P2- | AVALIVVPALAPEAAAKLIRDSSDQ | 262 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD343-P2- | IVAVALPALVAPEAAAKLIRDSSD | 263 |
| 1-32B | QRHFFTLSVKTQSGTKNLRIQGGGG | |
| | SGGGGSGGGGSGGGGSAYYIYSGGE | |
| | KIPLVLSRPLSSNEAAAKEAAAKEA | |
| | AAKMAEQSDKDVKYYTLEEIQKHKD | |
| | SKSTWLILHHKVYDLTKFLEEHPGG | |
| | EEVLGEQAGGDATENFEDVGHSTDA | |
| | RELSKTYIIGELHPDDRSKIAKPSE | |
| | TL | |
| aMTD385-P2- | IVAIAVPALVAPEAAAKLIRDSSDQ | 264 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD485-P2- | AILAAIVPLAVPEAAAKLIRDSSDQ | 265 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |
| aMTD830-P2- | IALVAAPVALVPEAAAKLIRDSSDQ | 266 |
| 1-32B | RHFFTLSVKTQSGTKNLRIQGGGGS | |
| | GGGGSGGGGSGGGGSAYYIYSGGEK | |
| | IPLVLSRPLSSNEAAAKEAAAKEAA | |
| | AKMAEQSDKDVKYYTLEEIQKHKDS | |
| | KSTWLILHHKVYDLTKFLEEHPGGE | |
| | EVLGEQAGGDATENFEDVGHSTDAR | |
| | ELSKTYIIGELHPDDRSKIAKPSET | |
| | L | |

The aMTD sequence of aMTD524-P2-1B, aMTD524-P2-1-24B and aMTD524-P2-1-32B were replaced to aMTD343, aMTD385, aMTD485 and aMTD830 for high-yield protein purification. aMTD830-P2-1B, aMTD830-P2-1-24B and aMTD830-P2-1-32B were selected because of high expression level in the supernatant (FIGS. 10b, 12b and 13b). aMTD830-P2-1B, aMTD830-P2-1-24B and aMTD830-P2-1-32B were homnogenous with single band in SDS-PAGE gel and a single peak in SE-HPLC analysis (FIGS. 10c, 12c and 13c). aMTD830-P2-1B, aMTD830-P2-1-24B and aMTD830-P2-1-32B overcame leptin resistance (FIGS. 10d, 12d and 13d). aMTD830-P2-1B, aMTD830-P2-1-24B and aMTD830-P2-1-32B were stable with thermal stress of 37° C. in serum free medium unlike instability of aMTD524-P2-1-6 and aMTD524-P2-1-24 in serum free medium (FIGS. 10e, 12e and 13e).

These results demonstrate the CP-SD recombinants, aMTD830-P2-1B, aMTD830-P2-1-24B and aMTD830-P2-1-32B are homogeneous, stable and biologically active proteins to overcome leptin resistance, which suggest aMTD830-P2-1B, aMTD830-P2-1-24B and aMTD830-P2-1-32B as possible therapeutics for obesity.

1. A cell-permeable truncated SOCS3 SH2 domain (CP-SD) recombinant protein, wherein the recombinant protein comprises:
i) a region of L69-Q96 in the SH2 domain of a human SOCS3 protein; and
ii) an advanced macromolecule transduction domain (aMTD),
wherein the aMTD has an amino acid sequence selected from the group consisting of SEQ ID Nos: 5-244.

2. The CP-SD recombinant protein according to claim 1,
wherein the recombinant protein further comprises one or more region(s) selected from the group consisting of a region of V120-M128, a region of H125-M128 and a region of A164-N185 in the SH2 domain of the human SOCS3 protein.

3. The CP-SD recombinant proteins according to claim 1, wherein the recombinant protein further comprises one or more solubilization domain (SD)(s).

4. The CP-SD recombinant protein according to claim 3,
wherein the recombinant protein is represented by any one of the following structural formulae:
A-B, B-A, A-B-C, A-C-B, B-A-C, B-C-A, C-A-B, C-B-A and A-C-B-C wherein A is an advanced macromolecule transduction domain (aMTD),
B is a truncated SOCS3 SH2 domain protein,
and C is a solubilization domain (SD).

5. The CP-SD recombinant protein according to claim 3,
wherein the recombinant protein has an amino acid sequence selected from the group consisting of SEQ ID NOs:251-266.

6. The CP-SD recombinant protein according to claim 3,
wherein the SD(s) have an amino acid sequence independently selected from the group consisting of SEQ ID Nos.: 248-249.

7. The CP-SD recombinant protein according to claim 1,
wherein the CP-SD recombinant protein is used for treating obesity.

8. A polynucleotide sequence encoding the CP-SD recombinant protein of claim 1.

9. A recombinant expression vector comprising the polynucleotide sequence of claim 8.

10. A transformant transformed with the recombinant expression vector of claim 9.

11. A composition comprising the CP-SD recombinant protein of claim 1 as an active ingredient.

12-18. (canceled)

19. A method of treating obesity related diseases in a subject comprising:
administering to the subject a therapeutically effective amount of the CP-SD recombinant protein of claim 1.

20. (canceled)

21. The method of treating obesity related diseases according to claim 12, wherein the obesity related diseases comprise obesity, depression, intracranial hypertension, dementia, heart attack, vascular sclerosis, irregular menstruation, cancer, arthritis, asthma, fatty liver, diabetes, hyperlipidemia, high blood pressure, gallbladder disease, coronary artery disease, gout, stroke.

22. A method of treating obesity related diseases in a subject comprising:
administering to the subject a therapeutically effective amount of the recombinant expression vector of claim 9.

23. A method of treating obesity related diseases in a subject comprising:
administering to the subject a therapeutically effective amount of the transformant transformed with the recombinant expression vector of claim 10.

24. A method of treating obesity related diseases in a subject comprising:
administering to the subject a therapeutically effective amount of the composition of claim 11.


SEQUENCE LISTING (283 sequences)

SEQ ID NO: 1 - protein, 28 aa, Artificial Sequence
  Amico acid sequence of human SOCS3
LIRDSSDQRHFFTLSVKTQSGTKNLRIQ

SEQ ID NO: 2 - protein, 9 aa, Artificial Sequence
  Amico acid sequence of human SOCS3
VLKLVHHYM

SEQ ID NO: 3 - protein, 4 aa, Artificial Sequence
  Amico acid sequence of human SOCS3
HHYM

SEQ ID NO: 4 - protein, 22 aa, Artificial Sequence
  Amico acid sequence of human SOCS3
AYYIYSGGEKIPLVLSRPLSSN

SEQ ID NO: 5 - protein, 12 aa, Artificial Sequence
  Amino acid Sequence of aMTD1
AAALAPVVLALP

SEQ ID NO: 6 - protein, 12 aa, Artificial Sequence
  Amino acid Sequence of aMTD2
AAAVPLLAVVVP

SEQ ID NO: 7 - protein, 12 aa, Artificial Sequence
  Amino acid Sequence of aMTD3
AALLVPAAVLAP

SEQ ID NO: 8 - protein, 12 aa, Artificial Sequence
  Amino acid Sequence of aMTD4
ALALLPVAALAP

SEQ ID NO: 9 - protein, 12 aa, Artificial Sequence
  Amino acid Sequence of aMTD5
AAALLPVALVAP

SEQ ID NO: 10 - protein, 12 aa, Artificial Sequence
  Amino acid Sequence of aMTD11
VVALAPALAALP

SEQ ID NO: 11 - protein, 12 aa, Artificial Sequence
  Amino acid Sequence of aMTD12
LLAAVPAVLLAP

SEQ ID NO: 12 - protein, 12 aa, Artificial Sequence
  Amino acid Sequence of aMTD13
AAALVPVVALLP

SEQ ID NO: 13 - protein, 12 aa, Artificial Sequence
  Amino acid Sequence of aMTD21
AVALLPALLAVP

SEQ ID NO: 14 - protein, 12 aa, Artificial Sequence
  Amino acid Sequence of aMTD22
AVVLVPVLAAAP

SEQ ID NO: 15 - protein, 12 aa, Artificial Sequence
  Amino acid Sequence of aMTD23
VVLVLPAAAAVP

SEQ ID NO: 16 - protein, 12 aa, Artificial Sequence
  Amino acid Sequence of aMTD24
IALAAPALIVAP

SEQ ID NO: 17 - protein, 12 aa, Artificial Sequence
  Amino acid Sequence of aMTD25
IVAVAPALVALP

SEQ ID NO: 18 - protein, 12 aa, Artificial Sequence
  Amino acid Sequence of aMTD42
VAALPVVAVVAP

SEQ ID NO: 19 - protein, 12 aa, Artificial Sequence
  Amino acid Sequence of aMTD43
LLAAPLVVAAVP

SEQ ID NO: 20 - protein, 12 aa, Artificial Sequence
  Amino acid Sequence of aMTD44
ALAVPVALLVAP

SEQ ID NO: 21 - protein, 12 aa, Artificial Sequence
  Amino acid Sequence of aMTD61
VAALPVLLAALP

SEQ ID NO: 22 - protein, 12 aa, Artificial Sequence
  Amino acid Sequence of aMTD62
VALLAPVALAVP

SEQ ID NO: 23 - protein, 12 aa, Artificial Sequence
  Amino acid Sequence of aMTD63
AALLVPALVAVP

SEQ ID NO: 24 - protein, 12 aa, Artificial Sequence
  Amino acid Sequence of aMTD64
AIVALPVAVLAP

SEQ ID NO: 25 - protein, 12 aa, Artificial Sequence
  Amino acid Sequence of aMTD65
IAIVAPVVALAP

SEQ ID NO: 26 - protein, 12 aa, Artificial Sequence
  Amino acid Sequence of aMTD81
AALLPALAALLP

SEQ ID NO: 27 - protein, 12 aa, Artificial Sequence
  Amino acid Sequence of aMTD82
AVVLAPVAAVLP

SEQ ID NO: 28 - protein, 12 aa, Artificial Sequence
  Amino acid Sequence of aMTD83
LAVAAPLALALP

SEQ ID NO: 29 - protein, 12 aa, Artificial Sequence
  Amino acid Sequence of aMTD84
AAVAAPLLLALP

SEQ ID NO: 30 - protein, 12 aa, Artificial Sequence
  Amino acid Sequence of aMTD85
LLVLPAAALAAP

SEQ ID NO: 31 - protein, 12 aa, Artificial Sequence
  Amino acid Sequence of aMTD101
LVALAPVAAVLP

SEQ ID NO: 32 - protein, 12 aa, Artificial Sequence
  Amino acid Sequence of aMTD102
LALAPAALALLP

SEQ ID NO: 33 - protein, 12 aa, Artificial Sequence
  Amino acid Sequence of aMTD103
ALIAAPILALAP

SEQ ID NO: 34 - protein, 12 aa, Artificial Sequence
  Amino acid Sequence of aMTD104
AVVAAPLVLALP

SEQ ID NO: 35 - protein, 12 aa, Artificial Sequence
  Amino acid Sequence of aMTD105
LLALAPAALLAP

SEQ ID NO: 36 - protein, 12 aa, Artificial Sequence
  Amino acid Sequence of aMTD121
AIVALPALALAP

SEQ ID NO: 37 - protein, 12 aa, Artificial Sequence
  Amino acid Sequence of aMTD123
AAIIVPAALLAP

SEQ ID NO: 38 - protein, 12 aa, Artificial Sequence
  Amino acid Sequence of aMTD124
IAVALPALIAAP

SEQ ID NO: 39 - protein, 12 aa, Artificial Sequence
  Amino acid Sequence of aMTD141
AVIVLPALAVAP

SEQ ID NO: 40 - protein, 12 aa, Artificial Sequence
  Amino acid Sequence of aMTD143
AVLAVPAVLVAP

SEQ ID NO: 41 - protein, 12 aa, Artificial Sequence
  Amino acid Sequence of aMTD144
VLAIVPAVALAP

SEQ ID NO: 42 - protein, 12 aa, Artificial Sequence
  Amino acid Sequence of aMTD145
LLAVVPAVALAP

SEQ ID NO: 43 - protein, 12 aa, Artificial Sequence
  Amino acid Sequence of aMTD161
AVIALPALIAAP

SEQ ID NO: 44 - protein, 12 aa, Artificial Sequence
  Amino acid Sequence of aMTD162
AVVALPAALIVP

SEQ ID NO: 45 - protein, 12 aa, Artificial Sequence
  Amino acid Sequence of aMTD163
LALVLPAALAAP

SEQ ID NO: 46 - protein, 12 aa, Artificial Sequence
  Amino acid Sequence of aMTD164
LAAVLPALLAAP

SEQ ID NO: 47 - protein, 12 aa, Artificial Sequence
  Amino acid Sequence of aMTD165
ALAVPVALAIVP

SEQ ID NO: 48 - protein, 12 aa, Artificial Sequence
  Amino acid Sequence of aMTD182
ALIAPVVALVAP

SEQ ID NO: 49 - protein, 12 aa, Artificial Sequence
  Amino acid Sequence of aMTD183
LLAAPVVIALAP

SEQ ID NO: 50 - protein, 12 aa, Artificial Sequence
  Amino acid Sequence of aMTD184
LAAIVPAIIAVP

SEQ ID NO: 51 - protein, 12 aa, Artificial Sequence
  Amino acid Sequence of aMTD185
AALVLPLIIAAP

SEQ ID NO: 52 - protein, 12 aa, Artificial Sequence
  Amino acid Sequence of aMTD201
LALAVPALAALP

SEQ ID NO: 53 - protein, 12 aa, Artificial Sequence
  Amino acid Sequence of aMTD204
LIAALPAVAALP

SEQ ID NO: 54 - protein, 12 aa, Artificial Sequence
  Amino acid Sequence of aMTD205
ALALVPAIAALP

SEQ ID NO: 55 - protein, 12 aa, Artificial Sequence
  Amino acid Sequence of aMTD221
AAILAPIVALAP

SEQ ID NO: 56 - protein, 12 aa, Artificial Sequence
  Amino acid Sequence of aMTD222
ALLIAPAAVIAP

SEQ ID NO: 57 - protein, 12 aa, Artificial Sequence
  Amino acid Sequence of aMTD223
AILAVPIAVVAP

SEQ ID NO: 58 - protein, 12 aa, Artificial Sequence
  Amino acid Sequence of aMTD224
ILAAVPIALAAP

SEQ ID NO: 59 - protein, 12 aa, Artificial Sequence
  Amino acid Sequence of aMTD225
VAALLPAAAVLP

SEQ ID NO: 60 - protein, 12 aa, Artificial Sequence
  Amino acid Sequence of aMTD241
AAAVVPVLLVAP

SEQ ID NO: 61 - protein, 12 aa, Artificial Sequence
  Amino acid Sequence of aMTD242
AALLVPALVAAP

SEQ ID NO: 62 - protein, 12 aa, Artificial Sequence
  Amino acid Sequence of aMTD243
AAVLLPVALAAP

SEQ ID NO: 63 - protein, 12 aa, Artificial Sequence
  Amino acid Sequence of aMTD245
AAALAPVLALVP

SEQ ID NO: 64 - protein, 12 aa, Artificial Sequence
  Amino acid Sequence of aMTD261
LVLVPLLAAAAP

SEQ ID NO: 65 - protein, 12 aa, Artificial Sequence
  Amino acid Sequence of aMTD262
ALIAVPAIIVAP

SEQ ID NO: 66 - protein, 12 aa, Artificial Sequence
  Amino acid Sequence of aMTD263
ALAVIPAAAILP

SEQ ID NO: 67 - protein, 12 aa, Artificial Sequence
  Amino acid Sequence of aMTD264
LAAAPVVIVIAP

SEQ ID NO: 68 - protein, 12 aa, Artificial Sequence
  Amino acid Sequence of aMTD265
VLAIAPLLAAVP

SEQ ID NO: 69 - protein, 12 aa, Artificial Sequence
  Amino acid Sequence of aMTD281
ALIVLPAAVAVP

SEQ ID NO: 70 - protein, 12 aa, Artificial Sequence
  Amino acid Sequence of aMTD282
VLAVAPALIVAP

SEQ ID NO: 71 - protein, 12 aa, Artificial Sequence
  Amino acid Sequence of aMTD283
AALLAPALIVAP

SEQ ID NO: 72 - protein, 12 aa, Artificial Sequence
  Amino acid Sequence of aMTD284
ALIAPAVALIVP

SEQ ID NO: 73 - protein, 12 aa, Artificial Sequence
  Amino acid Sequence of aMTD285
AIVLLPAAVVAP

SEQ ID NO: 74 - protein, 12 aa, Artificial Sequence
  Amino acid Sequence of aMTD301
VIAAPVLAVLAP

SEQ ID NO: 75 - protein, 12 aa, Artificial Sequence
  Amino acid Sequence of aMTD302
LALAPALALLAP

SEQ ID NO: 76 - protein, 12 aa, Artificial Sequence
  Amino acid Sequence of aMTD304
AIILAPIAAIAP

SEQ ID NO: 77 - protein, 12 aa, Artificial Sequence
  Amino acid Sequence of aMTD305
IALAAPILLAAP

SEQ ID NO: 78 - protein, 12 aa, Artificial Sequence
  Amino acid Sequence of aMTD321
IVAVALPALAVP

SEQ ID NO: 79 - protein, 12 aa, Artificial Sequence
  Amino acid Sequence of aMTD322
VVAIVLPALAAP

SEQ ID NO: 80 - protein, 12 aa, Artificial Sequence
  Amino acid Sequence of aMTD323
IVAVALPVALAP

SEQ ID NO: 81 - protein, 12 aa, Artificial Sequence
  Amino acid Sequence of aMTD324
IVAVALPAALVP

SEQ ID NO: 82 - protein, 12 aa, Artificial Sequence
  Amino acid Sequence of aMTD325
IVAVALPAVALP

SEQ ID NO: 83 - protein, 12 aa, Artificial Sequence
  Amino acid Sequence of aMTD341
IVAVALPAVLAP

SEQ ID NO: 84 - protein, 12 aa, Artificial Sequence
  Amino acid Sequence of aMTD342
VIVALAPAVLAP

SEQ ID NO: 85 - protein, 12 aa, Artificial Sequence
  Amino acid Sequence of aMTD343
IVAVALPALVAP

SEQ ID NO: 86 - protein, 12 aa, Artificial Sequence
  Amino acid Sequence of aMTD345
ALLIVAPVAVAP

SEQ ID NO: 87 - protein, 12 aa, Artificial Sequence
  Amino acid Sequence of aMTD361
AVVIVAPAVIAP

SEQ ID NO: 88 - protein, 12 aa, Artificial Sequence
  Amino acid Sequence of aMTD363
AVLAVAPALIVP

SEQ ID NO: 89 - protein, 12 aa, Artificial Sequence
  Amino acid Sequence of aMTD364
LVAAVAPALIVP

SEQ ID NO: 90 - protein, 12 aa, Artificial Sequence
  Amino acid Sequence of aMTD365
AVIVVAPALLAP

SEQ ID NO: 91 - protein, 12 aa, Artificial Sequence
  Amino acid Sequence of aMTD381
VVAIVLPAVAAP

SEQ ID NO: 92 - protein, 12 aa, Artificial Sequence
  Amino acid Sequence of aMTD382
AAALVIPAILAP

SEQ ID NO: 93 - protein, 12 aa, Artificial Sequence
  Amino acid Sequence of aMTD383
VIVALAPALLAP

SEQ ID NO: 94 - protein, 12 aa, Artificial Sequence
  Amino acid Sequence of aMTD384
VIVAIAPALLAP

SEQ ID NO: 95 - protein, 12 aa, Artificial Sequence
  Amino acid Sequence of aMTD385
IVAIAVPALVAP

SEQ ID NO: 96 - protein, 12 aa, Artificial Sequence
  Amino acid Sequence of aMTD401
AALAVIPAAILP

SEQ ID NO: 97 - protein, 12 aa, Artificial Sequence
  Amino acid Sequence of aMTD402
ALAAVIPAAILP

SEQ ID NO: 98 - protein, 12 aa, Artificial Sequence
  Amino acid Sequence of aMTD403
AAALVIPAAILP

SEQ ID NO: 99 - protein, 12 aa, Artificial Sequence
  Amino acid Sequence of aMTD404
LAAAVIPAAILP

SEQ ID NO: 100 - protein, 12 aa, Artificial Sequence
  Amino acid Sequence of aMTD405
LAAAVIPVAILP

SEQ ID NO: 101 - protein, 12 aa, Artificial Sequence
  Amino acid Sequence of aMTD421
AAILAAPLIAVP

SEQ ID NO: 102 - protein, 12 aa, Artificial Sequence
  Amino acid Sequence of aMTD422
VVAILAPLLAAP

SEQ ID NO: 103 - protein, 12 aa, Artificial Sequence
  Amino acid Sequence of aMTD424
AVVVAAPVLALP

SEQ ID NO: 104 - protein, 12 aa, Artificial Sequence
  Amino acid Sequence of aMTD425
AVVAIAPVLALP

SEQ ID NO: 105 - protein, 12 aa, Artificial Sequence
  Amino acid Sequence of aMTD442
ALAALVPAVLVP

SEQ ID NO: 106 - protein, 12 aa, Artificial Sequence
  Amino acid Sequence of aMTD443
ALAALVPVALVP

SEQ ID NO: 107 - protein, 12 aa, Artificial Sequence
  Amino acid Sequence of aMTD444
LAAALVPVALVP

SEQ ID NO: 108 - protein, 12 aa, Artificial Sequence
  Amino acid Sequence of aMTD445
ALAALVPALVVP

SEQ ID NO: 109 - protein, 12 aa, Artificial Sequence
  Amino acid Sequence of aMTD461
IAAVIVPAVALP

SEQ ID NO: 110 - protein, 12 aa, Artificial Sequence
  Amino acid Sequence of aMTD462
IAAVLVPAVALP

SEQ ID NO: 111 - protein, 12 aa, Artificial Sequence
  Amino acid Sequence of aMTD463
AVAILVPLLAAP

SEQ ID NO: 112 - protein, 12 aa, Artificial Sequence
  Amino acid Sequence of aMTD464
AVVILVPLAAAP

SEQ ID NO: 113 - protein, 12 aa, Artificial Sequence
  Amino acid Sequence of aMTD465
IAAVIVPVAALP

SEQ ID NO: 114 - protein, 12 aa, Artificial Sequence
  Amino acid Sequence of aMTD481
AIAIAIVPVALP

SEQ ID NO: 115 - protein, 12 aa, Artificial Sequence
  Amino acid Sequence of aMTD482
ILAVAAIPVAVP

SEQ ID NO: 116 - protein, 12 aa, Artificial Sequence
  Amino acid Sequence of aMTD483
ILAAAIIPAALP

SEQ ID NO: 117 - protein, 12 aa, Artificial Sequence
  Amino acid Sequence of aMTD484
LAVVLAAPAIVP

SEQ ID NO: 118 - protein, 12 aa, Artificial Sequence
  Amino acid Sequence of aMTD485
AILAAIVPLAVP

SEQ ID NO: 119 - protein, 12 aa, Artificial Sequence
  Amino acid Sequence of aMTD501
VIVALAVPALAP

SEQ ID NO: 120 - protein, 12 aa, Artificial Sequence
  Amino acid Sequence of aMTD502
AIVALAVPVLAP

SEQ ID NO: 121 - protein, 12 aa, Artificial Sequence
  Amino acid Sequence of aMTD503
AAIIIVLPAALP

SEQ ID NO: 122 - protein, 12 aa, Artificial Sequence
  Amino acid Sequence of aMTD504
LIVALAVPALAP

SEQ ID NO: 123 - protein, 12 aa, Artificial Sequence
  Amino acid Sequence of aMTD505
AIIIVIAPAAAP

SEQ ID NO: 124 - protein, 12 aa, Artificial Sequence
  Amino acid Sequence of aMTD521
LAALIVVPAVAP

SEQ ID NO: 125 - protein, 12 aa, Artificial Sequence
  Amino acid Sequence of aMTD522
ALLVIAVPAVAP

SEQ ID NO: 126 - protein, 12 aa, Artificial Sequence
  Amino acid Sequence of aMTD524
AVALIVVPALAP

SEQ ID NO: 127 - protein, 12 aa, Artificial Sequence
  Amino acid Sequence of aMTD525
ALAIVVAPVAVP

SEQ ID NO: 128 - protein, 12 aa, Artificial Sequence
  Amino acid Sequence of aMTD541
LLALIIAPAAAP

SEQ ID NO: 129 - protein, 12 aa, Artificial Sequence
  Amino acid Sequence of aMTD542
ALALIIVPAVAP

SEQ ID NO: 130 - protein, 12 aa, Artificial Sequence
  Amino acid Sequence of aMTD543
LLAALIAPAALP

SEQ ID NO: 131 - protein, 12 aa, Artificial Sequence
  Amino acid Sequence of aMTD544
IVALIVAPAAVP

SEQ ID NO: 132 - protein, 12 aa, Artificial Sequence
  Amino acid Sequence of aMTD545
VVLVLAAPAAVP

SEQ ID NO: 133 - protein, 12 aa, Artificial Sequence
  Amino acid Sequence of aMTD561
AAVAIVLPAVVP

SEQ ID NO: 134 - protein, 12 aa, Artificial Sequence
  Amino acid Sequence of aMTD562
ALIAAIVPALVP

SEQ ID NO: 135 - protein, 12 aa, Artificial Sequence
  Amino acid Sequence of aMTD563
ALAVIVVPALAP

SEQ ID NO: 136 - protein, 12 aa, Artificial Sequence
  Amino acid Sequence of aMTD564
VAIALIVPALAP

SEQ ID NO: 137 - protein, 12 aa, Artificial Sequence
  Amino acid Sequence of aMTD565
VAIVLVAPAVAP

SEQ ID NO: 138 - protein, 12 aa, Artificial Sequence
  Amino acid Sequence of aMTD582
VAVALIVPALAP

SEQ ID NO: 139 - protein, 12 aa, Artificial Sequence
  Amino acid Sequence of aMTD583
AVILALAPIVAP

SEQ ID NO: 140 - protein, 12 aa, Artificial Sequence
  Amino acid Sequence of aMTD585
ALIVAIAPALVP

SEQ ID NO: 141 - protein, 12 aa, Artificial Sequence
  Amino acid Sequence of aMTD601
AAILIAVPIAAP

SEQ ID NO: 142 - protein, 12 aa, Artificial Sequence
  Amino acid Sequence of aMTD602
VIVALAAPVLAP

SEQ ID NO: 143 - protein, 12 aa, Artificial Sequence
  Amino acid Sequence of aMTD603
VLVALAAPVIAP

SEQ ID NO: 144 - protein, 12 aa, Artificial Sequence
  Amino acid Sequence of aMTD604
VALIAVAPAVVP

SEQ ID NO: 145 - protein, 12 aa, Artificial Sequence
  Amino acid Sequence of aMTD605
VIAAVLAPVAVP

SEQ ID NO: 146 - protein, 12 aa, Artificial Sequence
  Amino acid Sequence of aMTD622
ALIVLAAPVAVP

SEQ ID NO: 147 - protein, 12 aa, Artificial Sequence
  Amino acid Sequence of aMTD623
VAAAIALPAIVP

SEQ ID NO: 148 - protein, 12 aa, Artificial Sequence
  Amino acid Sequence of aMTD625
ILAAAAAPLIVP

SEQ ID NO: 149 - protein, 12 aa, Artificial Sequence
  Amino acid Sequence of aMTD643
LALVLAAPAIVP

SEQ ID NO: 150 - protein, 12 aa, Artificial Sequence
  Amino acid Sequence of aMTD645
ALAVVALPAIVP

SEQ ID NO: 151 - protein, 12 aa, Artificial Sequence
  Amino acid Sequence of aMTD661
AAILAPIVAALP

SEQ ID NO: 152 - protein, 12 aa, Artificial Sequence
  Amino acid Sequence of aMTD664
ILIAIAIPAAAP

SEQ ID NO: 153 - protein, 12 aa, Artificial Sequence
  Amino acid Sequence of aMTD665
LAIVLAAPVAVP

SEQ ID NO: 154 - protein, 12 aa, Artificial Sequence
  Amino acid Sequence of aMTD666
AAIAIIAPAIVP

SEQ ID NO: 155 - protein, 12 aa, Artificial Sequence
  Amino acid Sequence of aMTD667
LAVAIVAPALVP

SEQ ID NO: 156 - protein, 12 aa, Artificial Sequence
  Amino acid Sequence of aMTD683
LAIVLAAPAVLP

SEQ ID NO: 157 - protein, 12 aa, Artificial Sequence
  Amino acid Sequence of aMTD684
AAIVLALPAVLP

SEQ ID NO: 158 - protein, 12 aa, Artificial Sequence
  Amino acid Sequence of aMTD685
ALLVAVLPAALP

SEQ ID NO: 159 - protein, 12 aa, Artificial Sequence
  Amino acid Sequence of aMTD686
AALVAVLPVALP

SEQ ID NO: 160 - protein, 12 aa, Artificial Sequence
  Amino acid Sequence of aMTD687
AILAVALPLLAP

SEQ ID NO: 161 - protein, 12 aa, Artificial Sequence
  Amino acid Sequence of aMTD703
IVAVALVPALAP

SEQ ID NO: 162 - protein, 12 aa, Artificial Sequence
  Amino acid Sequence of aMTD705
IVAVALLPALAP

SEQ ID NO: 163 - protein, 12 aa, Artificial Sequence
  Amino acid Sequence of aMTD706
IVAVALLPAVAP

SEQ ID NO: 164 - protein, 12 aa, Artificial Sequence
  Amino acid Sequence of aMTD707
IVALAVLPAVAP

SEQ ID NO: 165 - protein, 12 aa, Artificial Sequence
  Amino acid Sequence of aMTD724
VAVLAVLPALAP

SEQ ID NO: 166 - protein, 12 aa, Artificial Sequence
  Amino acid Sequence of aMTD725
IAVLAVAPAVLP

SEQ ID NO: 167 - protein, 12 aa, Artificial Sequence
  Amino acid Sequence of aMTD726
LAVAIIAPAVAP

SEQ ID NO: 168 - protein, 12 aa, Artificial Sequence
  Amino acid Sequence of aMTD727
VALAIALPAVLP

SEQ ID NO: 169 - protein, 12 aa, Artificial Sequence
  Amino acid Sequence of aMTD743
AIAIALVPVALP

SEQ ID NO: 170 - protein, 12 aa, Artificial Sequence
  Amino acid Sequence of aMTD744
AAVVIVAPVALP

SEQ ID NO: 171 - protein, 12 aa, Artificial Sequence
  Amino acid Sequence of aMTD746
VAIIVVAPALAP

SEQ ID NO: 172 - protein, 12 aa, Artificial Sequence
  Amino acid Sequence of aMTD747
VALLAIAPALAP

SEQ ID NO: 173 - protein, 12 aa, Artificial Sequence
  Amino acid Sequence of aMTD763
VAVLIAVPALAP

SEQ ID NO: 174 - protein, 12 aa, Artificial Sequence
  Amino acid Sequence of aMTD764
AVALAVLPAVVP

SEQ ID NO: 175 - protein, 12 aa, Artificial Sequence
  Amino acid Sequence of aMTD765
AVALAVVPAVLP

SEQ ID NO: 176 - protein, 12 aa, Artificial Sequence
  Amino acid Sequence of aMTD766
IVVIAVAPAVAP

SEQ ID NO: 177 - protein, 12 aa, Artificial Sequence
  Amino acid Sequence of aMTD767
IVVAAVVPALAP

SEQ ID NO: 178 - protein, 12 aa, Artificial Sequence
  Amino acid Sequence of aMTD783
IVALVPAVAIAP

SEQ ID NO: 179 - protein, 12 aa, Artificial Sequence
  Amino acid Sequence of aMTD784
VAALPAVALVVP

SEQ ID NO: 180 - protein, 12 aa, Artificial Sequence
  Amino acid Sequence of aMTD786
LVAIAPLAVLAP

SEQ ID NO: 181 - protein, 12 aa, Artificial Sequence
  Amino acid Sequence of aMTD787
AVALVPVIVAAP

SEQ ID NO: 182 - protein, 12 aa, Artificial Sequence
  Amino acid Sequence of aMTD788
AIAVAIAPVALP

SEQ ID NO: 183 - protein, 12 aa, Artificial Sequence
  Amino acid Sequence of aMTD803
AIALAVPVLALP

SEQ ID NO: 184 - protein, 12 aa, Artificial Sequence
  Amino acid Sequence of aMTD805
LVLIAAAPIALP

SEQ ID NO: 185 - protein, 12 aa, Artificial Sequence
  Amino acid Sequence of aMTD806
LVALAVPAAVLP

SEQ ID NO: 186 - protein, 12 aa, Artificial Sequence
  Amino acid Sequence of aMTD807
AVALAVPALVLP

SEQ ID NO: 187 - protein, 12 aa, Artificial Sequence
  Amino acid Sequence of aMTD808
LVVLAAAPLAVP

SEQ ID NO: 188 - protein, 12 aa, Artificial Sequence
  Amino acid Sequence of aMTD809
LIVLAAPALAAP

SEQ ID NO: 189 - protein, 12 aa, Artificial Sequence
  Amino acid Sequence of aMTD810
VIVLAAPALAAP

SEQ ID NO: 190 - protein, 12 aa, Artificial Sequence
  Amino acid Sequence of aMTD811
AVVLAVPALAVP

SEQ ID NO: 191 - protein, 12 aa, Artificial Sequence
  Amino acid Sequence of aMTD824
LIIVAAAPAVAP

SEQ ID NO: 192 - protein, 12 aa, Artificial Sequence
  Amino acid Sequence of aMTD825
IVAVIVAPAVAP

SEQ ID NO: 193 - protein, 12 aa, Artificial Sequence
  Amino acid Sequence of aMTD826
LVALAAPIIAVP

SEQ ID NO: 194 - protein, 12 aa, Artificial Sequence
  Amino acid Sequence of aMTD827
IAAVLAAPALVP

SEQ ID NO: 195 - protein, 12 aa, Artificial Sequence
  Amino acid Sequence of aMTD828
IALLAAPIIAVP

SEQ ID NO: 196 - protein, 12 aa, Artificial Sequence
  Amino acid Sequence of aMTD829
AALALVAPVIVP

SEQ ID NO: 197 - protein, 12 aa, Artificial Sequence
  Amino acid Sequence of aMTD830
IALVAAPVALVP

SEQ ID NO: 198 - protein, 12 aa, Artificial Sequence
  Amino acid Sequence of aMTD831
IIVAVAPAAIVP

SEQ ID NO: 199 - protein, 12 aa, Artificial Sequence
  Amino acid Sequence of aMTD832
AVAAIVPVIVAP

SEQ ID NO: 200 - protein, 12 aa, Artificial Sequence
  Amino acid Sequence of aMTD843
AVLVLVAPAAAP

SEQ ID NO: 201 - protein, 12 aa, Artificial Sequence
  Amino acid Sequence of aMTD844
VVALLAPLIAAP

SEQ ID NO: 202 - protein, 12 aa, Artificial Sequence
  Amino acid Sequence of aMTD845
AAVVIAPLLAVP

SEQ ID NO: 203 - protein, 12 aa, Artificial Sequence
  Amino acid Sequence of aMTD846
IAVAVAAPLLVP

SEQ ID NO: 204 - protein, 12 aa, Artificial Sequence
  Amino acid Sequence of aMTD847
LVAIVVLPAVAP

SEQ ID NO: 205 - protein, 12 aa, Artificial Sequence
  Amino acid Sequence of aMTD848
AVAIVVLPAVAP

SEQ ID NO: 206 - protein, 12 aa, Artificial Sequence
  Amino acid Sequence of aMTD849
AVILLAPLIAAP

SEQ ID NO: 207 - protein, 12 aa, Artificial Sequence
  Amino acid Sequence of aMTD850
LVIALAAPVALP

SEQ ID NO: 208 - protein, 12 aa, Artificial Sequence
  Amino acid Sequence of aMTD851
VLAVVLPAVALP

SEQ ID NO: 209 - protein, 12 aa, Artificial Sequence
  Amino acid Sequence of aMTD852
VLAVAAPAVLLP

SEQ ID NO: 210 - protein, 12 aa, Artificial Sequence
  Amino acid Sequence of aMTD863
AAVVLLPIIAAP

SEQ ID NO: 211 - protein, 12 aa, Artificial Sequence
  Amino acid Sequence of aMTD864
ALLVIAPAIAVP

SEQ ID NO: 212 - protein, 12 aa, Artificial Sequence
  Amino acid Sequence of aMTD865
AVLVIAVPAIAP

SEQ ID NO: 213 - protein, 12 aa, Artificial Sequence
  Amino acid Sequence of aMTD867
ALLVVIAPLAAP

SEQ ID NO: 214 - protein, 12 aa, Artificial Sequence
  Amino acid Sequence of aMTD868
VLVAAILPAAIP

SEQ ID NO: 215 - protein, 12 aa, Artificial Sequence
  Amino acid Sequence of aMTD870
VLVAAVLPIAAP

SEQ ID NO: 216 - protein, 12 aa, Artificial Sequence
  Amino acid Sequence of aMTD872
VLAAAVLPLVVP

SEQ ID NO: 217 - protein, 12 aa, Artificial Sequence
  Amino acid Sequence of aMTD875
AIAIVVPAVAVP

SEQ ID NO: 218 - protein, 12 aa, Artificial Sequence
  Amino acid Sequence of aMTD877
VAIIAVPAVVAP

SEQ ID NO: 219 - protein, 12 aa, Artificial Sequence
  Amino acid Sequence of aMTD878
IVALVAPAAVVP

SEQ ID NO: 220 - protein, 12 aa, Artificial Sequence
  Amino acid Sequence of aMTD879
AAIVLLPAVVVP

SEQ ID NO: 221 - protein, 12 aa, Artificial Sequence
  Amino acid Sequence of aMTD881
AALIVVPAVAVP

SEQ ID NO: 222 - protein, 12 aa, Artificial Sequence
  Amino acid Sequence of aMTD882
AIALVVPAVAVP

SEQ ID NO: 223 - protein, 12 aa, Artificial Sequence
  Amino acid Sequence of aMTD883
LAIVPAAIAALP

SEQ ID NO: 224 - protein, 12 aa, Artificial Sequence
  Amino acid Sequence of aMTD885
LVAIAPAVAVLP

SEQ ID NO: 225 - protein, 12 aa, Artificial Sequence
  Amino acid Sequence of aMTD887
VLAVAPAVAVLP

SEQ ID NO: 226 - protein, 12 aa, Artificial Sequence
  Amino acid Sequence of aMTD888
ILAVVAIPAAAP

SEQ ID NO: 227 - protein, 12 aa, Artificial Sequence
  Amino acid Sequence of aMTD889
ILVAAAPIAALP

SEQ ID NO: 228 - protein, 12 aa, Artificial Sequence
  Amino acid Sequence of aMTD891
ILAVAAIPAALP

SEQ ID NO: 229 - protein, 12 aa, Artificial Sequence
  Amino acid Sequence of aMTD893
VIAIPAILAAAP

SEQ ID NO: 230 - protein, 12 aa, Artificial Sequence
  Amino acid Sequence of aMTD895
AIIIVVPAIAAP

SEQ ID NO: 231 - protein, 12 aa, Artificial Sequence
  Amino acid Sequence of aMTD896
AILIVVAPIAAP

SEQ ID NO: 232 - protein, 12 aa, Artificial Sequence
  Amino acid Sequence of aMTD897
AVIVPVAIIAAP

SEQ ID NO: 233 - protein, 12 aa, Artificial Sequence
  Amino acid Sequence of aMTD899
AVVIALPAVVAP

SEQ ID NO: 234 - protein, 12 aa, Artificial Sequence
  Amino acid Sequence of aMTD900
ALVAVIAPVVAP

SEQ ID NO: 235 - protein, 12 aa, Artificial Sequence
  Amino acid Sequence of aMTD901
ALVAVLPAVAVP

SEQ ID NO: 236 - protein, 12 aa, Artificial Sequence
  Amino acid Sequence of aMTD902
ALVAPLLAVAVP

SEQ ID NO: 237 - protein, 12 aa, Artificial Sequence
  Amino acid Sequence of aMTD904
AVLAVVAPVVAP

SEQ ID NO: 238 - protein, 12 aa, Artificial Sequence
  Amino acid Sequence of aMTD905
AVIAVAPLVVAP

SEQ ID NO: 239 - protein, 12 aa, Artificial Sequence
  Amino acid Sequence of aMTD906
AVIALAPVVVAP

SEQ ID NO: 240 - protein, 12 aa, Artificial Sequence
  Amino acid Sequence of aMTD907
VAIALAPVVVAP

SEQ ID NO: 241 - protein, 12 aa, Artificial Sequence
  Amino acid Sequence of aMTD908
VALALAPVVVAP

SEQ ID NO: 242 - protein, 12 aa, Artificial Sequence
  Amino acid Sequence of aMTD910
VAALLPAVVVAP

SEQ ID NO: 243 - protein, 12 aa, Artificial Sequence
  Amino acid Sequence of aMTD911
VALALPAVVVAP

SEQ ID NO: 244 - protein, 12 aa, Artificial Sequence
  Amino acid Sequence of aMTD912
VALLAPAVVVAP

SEQ ID NO: 245 - protein, 5 aa, Artificial Sequence
  Amino acid Sequence of linker
EAAAK

SEQ ID NO: 246 - protein, 5 aa, Artificial Sequence
  Amino acid Sequence of linker
GGGGS

SEQ ID NO: 247 - protein, 4 aa, Artificial Sequence
  Amino acid Sequence of linker
GSGS

SEQ ID NO: 248 - protein, 99 aa, Artificial Sequence
  Amino acid Sequence of SD
MAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 249 - protein, 100 aa, Artificial Sequence
  Amino acid Sequence of SD
GSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 250 - protein, 20 aa, Artificial Sequence
  Amino acid Sequence of histidine tag
GSSHHHHHHSSGLVPRGSHM

SEQ ID NO: 251 - protein, 159 aa, Artificial Sequence
  Amino acid Sequence of aMTD524-P2-1B
AVALIVVPALAPEAAAKLIRDSSDQRHFFTLSVKTQSGTKNLRIQEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 252 - protein, 159 aa, Artificial Sequence
  Amino acid Sequence of aMTD343-P2-1B
IVAVALPALVAPEAAAKLIRDSSDQRHFFTLSVKTQSGTKNLRIQEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 253 - protein, 159 aa, Artificial Sequence
  Amino acid Sequence of aMTD385-P2-1B
IVAIAVPALVAPEAAAKLIRDSSDQRHFFTLSVKTQSGTKNLRIQEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 254 - protein, 159 aa, Artificial Sequence
  Amino acid Sequence of aMTD485-P2-1B
AILAAIVPLAVPEAAAKLIRDSSDQRHFFTLSVKTQSGTKNLRIQEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 255 - protein, 159 aa, Artificial Sequence
  Amino acid Sequence of aMTD830-P2-1B
IALVAAPVALVPEAAAKLIRDSSDQRHFFTLSVKTQSGTKNLRIQEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 256 - protein, 160 aa, Artificial Sequence
  Amino acid Sequence of aMTD524-P2-1E
AVALIVVPALAPEAAAKLIRDSSDQRHFFTLSVKTQSGTKNLRIQEAAAKEAAAKEAAAKGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 257 - protein, 213 aa, Artificial Sequence
  Amino acid Sequence of aMTD524-P2-1-24B
AVALIVVPALAPEAAAKLIRDSSDQRHFFTLSVKTQSGTKNLRIQGGGGSGGGGSGGGGSGGGGSHHYMGSGSGSGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 258 - protein, 213 aa, Artificial Sequence
  Amino acid Sequence of aMTD343-P2-1-24B
IVAVALPALVAPEAAAKLIRDSSDQRHFFTLSVKTQSGTKNLRIQGGGGSGGGGSGGGGSGGGGSHHYMGSGSGSGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 259 - protein, 213 aa, Artificial Sequence
  Amino acid Sequence of aMTD385-P2-1-24B
IVAIAVPALVAPEAAAKLIRDSSDQRHFFTLSVKTQSGTKNLRIQGGGGSGGGGSGGGGSGGGGSHHYMGSGSGSGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 260 - protein, 213 aa, Artificial Sequence
  Amino acid Sequence of aMTD485-P2-1-24B
AILAAIVPLAVPEAAAKLIRDSSDQRHFFTLSVKTQSGTKNLRIQGGGGSGGGGSGGGGSGGGGSHHYMGSGSGSGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 261 - protein, 213 aa, Artificial Sequence
  Amino acid Sequence of aMTD830-P2-1-24B
IALVAAPVALVPEAAAKLIRDSSDQRHFFTLSVKTQSGTKNLRIQGGGGSGGGGSGGGGSGGGGSHHYMGSGSGSGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 262 - protein, 201 aa, Artificial Sequence
  Amino acid Sequence of aMTD524-P2-1-32B
AVALIVVPALAPEAAAKLIRDSSDQRHFFTLSVKTQSGTKNLRIQGGGGSGGGGSGGGGSGGGGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 263 - protein, 201 aa, Artificial Sequence
  Amino acid Sequence of aMTD343-P2-1-32B
IVAVALPALVAPEAAAKLIRDSSDQRHFFTLSVKTQSGTKNLRIQGGGGSGGGGSGGGGSGGGGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 264 - protein, 201 aa, Artificial Sequence
  Amino acid Sequence of aMTD385-P2-1-32B
IVAIAVPALVAPEAAAKLIRDSSDQRHFFTLSVKTQSGTKNLRIQGGGGSGGGGSGGGGSGGGGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 265 - protein, 201 aa, Artificial Sequence
  Amino acid Sequence of aMTD485-P2-1-32B
AILAAIVPLAVPEAAAKLIRDSSDQRHFFTLSVKTQSGTKNLRIQGGGGSGGGGSGGGGSGGGGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 266 - protein, 201 aa, Artificial Sequence
  Amino acid Sequence of aMTD830-P2-1-32B
IALVAAPVALVPEAAAKLIRDSSDQRHFFTLSVKTQSGTKNLRIQGGGGSGGGGSGGGGSGGGGSAYYIYSGGEKIPLVLSRPLSSNEAAAKEAAAKEAAAKMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 267 - protein, 45 aa, Artificial Sequence
  Amino acid Sequence of aMTD524-P2-1
AVALIVVPALAPEAAAKLIRDSSDQRHFFTLSVKTQSGTKNLRIQ

SEQ ID NO: 268 - protein, 65 aa, Artificial Sequence
  Amino acid Sequence of aMTD524-P2-1-6
GSSHHHHHHSSGLVPRGSHMAVALIVVPALAPEAAAKLIRDSSDQRHFFTLSVKTQSGTKNLRIQ

SEQ ID NO: 269 - protein, 35 aa, Artificial Sequence
  Amino acid Sequence of aMTD524-P8
AVALIVVPALAPGTFLIRDSGGGGSGTKNLRIQCE

SEQ ID NO: 270 - protein, 40 aa, Artificial Sequence
  Amino acid Sequence of aMTD524-P8-1
AVALIVVPALAPGGGGSGTFLIRDSGGGGSGTKNLRIQCE

SEQ ID NO: 271 - protein, 41 aa, Artificial Sequence
  Amino acid Sequence of aMTD524-P9
AVALIVVPALAPGTFLIRDSGGGGSGGGGGSGTKNLRIQCE

SEQ ID NO: 272 - protein, 20 aa, Artificial Sequence
  Amino acid Sequence of aMTD524-P10
AVALIVVPALAPGTFLIRDS

SEQ ID NO: 273 - protein, 25 aa, Artificial Sequence
  Amino acid Sequence of aMTD524-P10-1
AVALIVVPALAPGGGGSGTFLIRDS

SEQ ID NO: 274 - protein, 22 aa, Artificial Sequence
  Amino acid Sequence of aMTD524-P11
AVALIVVPALAPGTKNLRIQCE

SEQ ID NO: 275 - protein, 27 aa, Artificial Sequence
  Amino acid Sequence of aMTD524-P11-1
AVALIVVPALAPGGGGSGTKNLRIQCE

SEQ ID NO: 276 - protein, 33 aa, Artificial Sequence
  Amino acid Sequence of aMTD524-P12
AVALIVVPALAPGTFLIRDSSDQRHFFTLSVKT

SEQ ID NO: 277 - protein, 32 aa, Artificial Sequence
  Amino acid Sequence of aMTD524-P13
AVALIVVPALAPFFTLSVKTQSGTKNLRIQCE

SEQ ID NO: 278 - protein, 34 aa, Artificial Sequence
  Amino acid Sequence of aMTD524-P14
AVALIVVPALAPTGGEANLLLSAEPAGTFLIRDS

SEQ ID NO: 279 - protein, 130 aa, Artificial Sequence
  Amino acid Sequence of aMTD-P2-1-21
TGGEANLLLSAGSGSGSGSGSGSGSGSAVALIVVPALAPEAAAKLIRDSSDQRHFFTLSVKTQSGTKNLRIQGGGGSGGGGSGGGGSGGGGSVLKLVHHYMGSGSGSGSAYYIYSGGEKIPLVLSRPLSS

SEQ ID NO: 280 - protein, 99 aa, Artificial Sequence
  Amino acid Sequence of aMTD-P2-1-23
AVALIVVPALAPEAAAKLIRDSSDQRHFFTLSVKTQSGTKNLRIQEAAAKEAAAKEAAAKEAAAKHHYMGSGSGSGSAYYIYSGGEKIPLVLSRPLSSN

SEQ ID NO: 281 - protein, 99 aa, Artificial Sequence
  Amino acid Sequence of aMTD-P2-1-24
AVALIVVPALAPEAAAKLIRDSSDQRHFFTLSVKTQSGTKNLRIQGGGGSGGGGSGGGGSGGGGSHHYMGSGSGSGSAYYIYSGGEKIPLVLSRPLSSN

SEQ ID NO: 282 - protein, 131 aa, Artificial Sequence
  Amino acid Sequence of aMTD524-36
AVALIVVPALAPEAAAKGFYWSAVTGGEANLLLSAEPAGTFLIRDSSDQRHFFTLSVKTQSGTKNLRIQCEGGSFSLQSDPRSTQPVPRFDCVLKLVHHYMGSGSGSGSAYYIYSGGEKIPLVLSRPLSSN

SEQ ID NO: 283 - protein, 77 aa, Artificial Sequence
  Amino acid Sequence of aMTD524-37
AVALIVVPALAPEAAAKLIRDSSDQRHFFTLSVKTQSGTKNLRIQCEGGSFSLQSDPRSTQPVPRFDCVLKLVHHYM